(19) United States
(12) Patent Application Publication
(10) Pub. No.: US 2007/0276009 A1
(43) Pub. Date: Nov. 29, 2007

(54) Compositions and Methods for Viral Inhibition

(76) Inventors: Zhi-Jie Ni, Fremont, CA (US); Bryan Chang, Cambridge, MA (US); Weibo Wang, Moraga, CA (US); Amy Weiner, Emeryville, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 10/576,045

(22) PCT Filed: Oct. 15, 2004
(86) PCT No.: PCT/US04/34169

§ 371 Date: Apr. 25, 2007

Related U.S. Application Data
(60) Provisional application No. 60/511,769, filed on Oct. 15, 2003.

(51) Int. Cl. A61K 31/403; C07D 209/82

(52) U.S. Cl. 514/339; 514/411; 548/444

(57) ABSTRACT

The present invention discloses methods and compositions for viral inhibition, particularly inhibition of HCV and SARS. The invention also provides compositions including carbazole derivatives useful for viral inhibition.

### CROSS REFERENCE TO RELATED APPLICATION

This application claims the benefit of U.S. patent application No. 60/511,769 filed on Oct. 15, 2003. The disclosure of the above provisional application is herein incorporated by reference in its entirety and for all purposes as if fully set forth herein.

### FIELD OF INVENTION

The present invention is directed to novel methods and compositions for viral inhibition. In some embodiments, methods are provided for inhibition of HCV and SARS. The invention also is directed to compositions including novel carbazole derivatives useful for viral inhibition.

### BACKGROUND OF THE INVENTION

Hepatitis is a systemic disease, which predominantly affects the liver. The disease is typified by the initial onset of symptoms such as anorexia, nausea, vomiting, fatigue, malaise, artlralgias, myalgias, and headaches, followed by the onset of jaundice. The disease may also be characterized by increased serum levels of the aminotransferases AST and ALT. Quantification of these enzymes in serum indicates the extent of liver damage.

There are five general categories of viral agents which have been associated with hepatitis: the hepatitis A virus (HAV); the hepatitis B virus (HBV); two types of non-A, non-B (NANB) agents, one blood-borne (hepatitis C) and the other enterically transmitted (hepatitis E); and the HBV-associated delta agent (hepatitis D).

There are two general clinical categories of hepatitis, acute hepatitis and chronic hepatitis. Symptoms for acute hepatitis range from asymptomatic and non-apparent to fatal infections. The disease may be subclinical and persistent, or rapidly progress to chronic liver disease with cirrhosis, and in some cases, to hepatocellular carcinoma. Acute hepatitis B infection in adult Caucasians in the United States progresses to chronic hepatitis B in about 5% to 10% of the cases. In the remainder of the cases, approximately 65% are asymptomatic. In the Far East, infection is usually perinatal, and 50% to 90% progress to the chronic state. It is likely that the different rates of progression are linked to the age at infection rather than genetic differences in the hosts. In the United States, about 0.2% of the population is chronically infected, with higher percentages in high-risk groups such as physicians, drug addicts and renal dialysis patients. In countries such as Taiwan, Hong Kong and Singapore, the level in the population with hepatitis infection may be as high as 10%.

In the United States, about 20% of patients with chronic hepatitis die of liver failure, and a further 5% develop hepatitis B-associated carcinoma. In the Far East, a large percentage of the population is infected with HBV, and after a long chronic infection (20 to 40 years), approximately 25% of these will develop hepatocellular carcinoma.

After the development of serologic tests for both hepatitis A and B, investigators identified other patients with hepatitis-like symptoms, and with incubation periods and modes of transmission consistent with an infectious disease, but without serologic evidence of hepatitis A or B infection. After almost 15 years, the causative agent was identified as an RNA virus. This virus (designated “hepatitis C”) has no homology with HBV, retroviruses, or other hepatitis viruses.

Hepatitis C (HCV) appears to be the major cause of post-transfusion and sporadic non-A, non-B (NANB) hepatitis worldwide, and plays a major role in the development of chronic liver disease, including hepatocellular carcinoma (Kuo et al., Science 244:362-364, 1989; Choo et al., British Medical Bulletin 46(2):423-441, 1990). Of the approximately 3 million persons who receive transfusions each year, approximately 150,000 will develop acute hepatitis C (Davis et al., New Eng. J. Med. 321(22):1501-1506, 1989). In addition, of those that develop acute hepatitis C, at least one-half will develop chronic hepatitis C.

Until recently, no therapy has proven effective for treatment of acute or chronic hepatitis B or C infections, and patients infected with hepatitis must generally allow the disease to run its course. Most anti-viral drugs, such as acyclovir, as well as attempts to bolster the immune system through the use of corticosteroids have proven ineffective (Alter, “Viral hepatitis and liver disease,” Zuckerman (ed.), New York: Alan R. Liss, pp. 537-42, 1988). Some anti-viral activity has been observed with adenosine arabinoside (Jacyna et al., British Med. Bull. 46:368-382, 1990), although toxic side effects, which are associated with this drug render such treatment unacceptable.

One treatment that has provided some benefit for chronic hepatitis B and C infections is the use of recombinant alpha interferon (Davis et al., New Eng. J. Med. 321(22):1501-1506, 1989; Perrillo et al., New Eng. J. Med. 323:295-301, 1990). However, for patients with hepatitis B infections only about 35% of infectees responded to such treatment, and in perinatal infectees only about 10% responded to treatment. For hepatitis C infections, despite apparent short-term success utilizing such therapy, six months after termination of treatment half of the patients who responded to therapy had relapsed. In addition, a further difficulty with alpha interferon therapy is that the composition frequently has toxic side effects such as nausea, and flu-like symptoms, which require reduced dosages for sensitive patients.

Hepatocellular carcinoma is a disease that is related to hepatitis B and hepatitis C infections. Briefly, hepatocellular carcinoma is the most common cancer worldwide. It is responsible for approximately 1,000,000 deaths annually, most of them in China and in sub-Saharan Africa. There is strong evidence of an etiologic role for hepatitis B infection in hepatocellular carcinoma. Carriers of the HBV are at greater than 90 times higher risk for the development of hepatocellular carcinoma than noncarriers. In many cases, hepatitis B virus DNA is integrated within the cellular genome of the tumor. Similarly, hepatitis C virus has also recently been found to be associated with hepatocellular carcinoma, based upon the observation that circulating HCV antibodies can be found in some patients with hepatocellular carcinoma. At present, surgical resection offers the only treatment for hepatocellular carcinoma, as chemotherapy, radiotherapy, and immunotherapy have not shown much promise (Colombo et al., Lancet 1006-1008, 1989; Bisceglie et al., Ann. of Internal Med. 108:390-401, 1988; Watanabe et al., Int. J. Cancer 48:340-343, 1991; Bisceglie et al., Amer. J. Gastro. 86:335-338, 1991).

Severe Acute Respiratory Syndrome, or “SARS”, is an often fatal respiratory illness that has recently been reported in Asia, North America, and Europe. The agent responsible for SARS has recently been posited to be a previously unrecognized coronavirus, which has recently been sequenced by the Centers for Disease Control and Prevention (CDC).

Given the severe threat to humans posed by viral infections such as HCV and SARS, it is clear that new therapies for treating such infections are critical importance. This invention is directed to these, as well as other, important ends.

### SUMMARY OF THE INVENTION

In some embodiments, the present invention provides methods for treating a viral infection in a patient suffering therefrom, comprising administering to said patient a therapeutically effective amount of a substituted carbazole. In some embodiments, the substituted carbazole is a compound of Formula I: 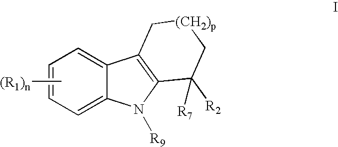
wherein:
each R₁ is independently
a. H, halogen, formyl, carbamoyl, carbamoylamino, carbamoyloxy, NO₂, amino, azido, hydrazino, hydroxylamino, sulfoxyl, sulfonyl, sulfide, disulfide, an ether having 2 to 10 carbon atoms and 1 to 4 oxygen or sulfur atoms;
b. alkyl, alkenyl, alkynyl, perhaloalkyl, alkoxy, alkoxyalkyl, —C(═O)alkyl, —OC(═O)alkyl, —C(═O)alkoxy, alkylsulfonyl, —C(═O)alkylamino, —C(═O)alkylaminoalkyl, —C(═O)NR₄R₅, —C(═O)NR₄R₆, —NHC(═O)R₇, —C(═O)R₈, monoalkylaminoalkyl, dialkylaminoalkyl, perhaloalkoxy, S-alkyl, urea optionally substituted with aryl wherein said aryl is optionally substituted with up to three halogen atoms;
c. heterocycloalkyl, heterocycloalkylamino, heterocycloalkylaminoalkyl, heterocycloalkylalkyl, monoalkylaminoalkyl, dialkylaminoalkyl, alkenylaminoalkyl, alkoxyalkylaminoalkyl, heterocycloalkylalkylaminoalkyl,
d. aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl; or
e. heteroaryl, heteroarylalkyl, alkylheteroaryl, heteroarylalkylamino, heteroarylalkylaminoalkyl, arylalkyloxy or arylsulfonyl optionally substituted with up to three groups selected from CN, halogen and alkyl;
wherein any of the foregoing groups can be independently substituted with up to three groups selected from formyl, OH, halogen, C₁₋₆ alkoxy, amino, monoalkylamino, dialkylamino, hydroxyalkyl, arylalkyl, alkyl, aryl, heteroaryl, alkenyl, alkynyl, heteroarylalkyl, CN, perhaloalkyl, monoalkylaminoalkyl, dialkylaminoalkyl, thiol, thioalkoxy, carboxyl, amido, amidino, NO₂, NO₃, perhaloalkoxy, S-alkyl, arylalkyloxy, S-arylalkyl, azido, hydrazino, hydroxylamino, sulfoxyl, sulfonyl, sulfide, disulfide, aryl optionally substituted with up to three halogen atoms, and urea optionally substituted with aryl wherein said aryl is optionally substituted with up to three halogen atoms;
n is 1 to 4;
p is 0 to 2;
R₄ is H, alkyl optionally substituted with C₁₋₆ alkoxy, allyl, alkoxyalkyl, heterocycloalkylalkyl, arylalkyl optionally substituted with up to three groups selected from dialkylamino, C₁₋₆ alkoxy, perhaloalkyl and halogen, heteroarylalkyl, monoalkylaminoalkyl, or dialkylaminoalkyl; wherein said alkyl is optionally substituted with C₁₋₆ alkoxy; and said arylalkyl is optionally substituted with up to three groups selected from dialkylamino, C₁₋₆ alkoxy, perhaloalkyl and halogen;
R₅ is H or alkyl;
or R₄ and R₅, together with the nitrogen atom to which they are attached, can form a heterocycloalkyl ring which can optionally be substituted with up to three alkyl groups;
R₇ and R₈ are independently H, NH₂, alkyl, alkoxy, aryl, heteroaryl, arylalkyl, heteroarylalkyl or heterocycloalkyl, wherein said aryl group can optionally be substituted with up to three groups selected from alkoxy, alkyl, perhaloalkyl, halogen and aryl;
R₂ is heteroaryl, arylalkyl, alkyl, formyl, —C(═O)NH₂, or —NHR₆;
R₆ is H, formyl, alkyl, alkenyl, alkynyl, arylalkyl, heterocycloalkyl, alkylsulfonyl, arylsulfonyl, —C(═O)NH₂, —C(═O)-alkyl, heteroarylalkyl, —C(═O)-alkylaminoalkyl, —C(═O)-aryl, arylalkanoylalkyl, heterocycloalkylalkyl, aryloxyalkyl, monoalkylaminoalkyl, dialkylaminoalkyl, allyl or urea;
wherein:
said alkyl, alkenyl or alkynyl groups can be optionally substituted with up to three groups selected from OH, halogen and C₁₋₆ alkoxy;
said arylalkyl is optionally substituted with up to three groups selected from OH, alkyl, perlialoalkyl, halogen, C₁₋₆ alkoxy, monoalkylamino, dialkylamino and hydroxyalkyl;
said heterocycloalkyl is optionally substituted with up to three groups selected from arylalkyl, alkyl, OH, halogen and C₁₋₆ alkoxy;
said arylsulfonyl is optionally substituted with up to three groups selected from CN, halogen, alkyl, OH, C₁₋₆ alkoxy, monoalkylamino, dialkylamino and hydroxyalkyl;
said —C(═O)-alkyl is optionally substituted with up to three groups selected from OH, halogen, perhaloalkyl and C₁₋₆ alkoxy;
said —C(═O)-aryl is optionally substituted with up to three groups selected from OH, alkyl, perhaloalkyl, halogen, C₁₋₆ alkoxy, monoalkylamino, dialkylamino and hydroxyalkyl
said heterocycloalkylalkyl is optionally substituted with up to three groups selected from OH, arylalkyl, alkyl, halogen and C₁₋₆ alkoxy;
said aryloxyalkyl is optionally substituted with up to three groups selected from OH, halogen, C₁₋₆ alkoxy, monoalkylamino, dialkylamino and hydroxyalkyl; and
said urea is optionally substituted with aryl, wherein said aryl is optionally substituted with up to three groups selected from OH, halogen, C₁₋₆ alkoxy, monoalkylamino, dialkylamino and hydroxyalkyl; and
R₉ is H or alkyl.

In some embodiments, an R₁ group is present at the 6-position of the substituted carbazole. In some further embodiments, the substituted carbazole contains a single R₁ group. In some further embodiments, the substituted carbazole contains a single R₁ group at the 6-position thereof.

In some embodiments, R₁ is —C(═O)NR₄R₅. hi further embodiments, R₂ is NHR₆. In still further embodiments, R₁ is —C(═O)NR₄R₅ and R₂ is NHR₆. In further embodiments, R₂ is —NHR₆ wherein R₆ is cycloalkyl.

In some embodiments wherein R₁ is —C(═O)NR₄R₅ and R₂ is NHR₆, R₄ is H, alkyl, allyl, alkoxyalkyl, heterocycloalkylalkyl, arylalkyl, heteroarylalkyl, monoalkylaminoalkyl or dialkylaminoalkyl, wherein said arylalkyl can be optionally substituted with up to three groups selected from halogen, haloalkyl, perhaloalkyl, C₁₋₆ alkoxy and dialkylamino.

In further embodiments wherein R₁ is —C(═O)NR₄R₅ and R₂ is NHR₆, R₆ is alkyl, arylalkyl optionally substituted with up to three halogen atoms, heteroarylalkyl, N-alkanoylaminoalkyl, or heterocycloalkylalkyl.

In further embodiments wherein R₁ is —C(═O)NR₄R₅ and R₂ is NHR₆, R₆ is alkyl, arylalkyl optionally substituted with up to three groups selected from halogen and C₁₋₆ alkoxy, heteroarylalkyl, N-alkanoylamminoalkyl, or heterocycloalkylalkyl.

In some embodiments, R₁ is —C(═O)NR₄R₅, where R₄ is alkyl, heteroarylalkyl, or heterocycloalkylalkyl.

In fiurther embodiments, R₂ is NHR₆, where R₆ is alkyl, arylalkyl optionally substituted with to up to three groups selected from halogen and C₁₋₆ alkoxy, heteroarylalkyl, or N-alkanoylaminoalkyl.

In further embodiments, R₁ is —C(═O)NR₄R₅, where R₄ is alkyl, heteroarylalkyl, or heterocycloalkylalkyl; and R₂ is NHR₆, where R₆ is alkyl, arylalkyl optionally substituted with up to three halogen atoms, heteroarylalkyl, or N-alkanoylaminoalkyl. In some such embodiments, R₄ is heteroarylalkyl and R₆ is alkyl or arylalkyl optionally substituted with up to three halogen atoms, preferably wherein said arylalkyl is phenylalkyl. In some such embodiments, R₄ is heterocycloalkylalkyl, and R₆ is alkyl, preferably wherein said heterocycloalkylalkyl is pyrrolidino-alkyl.

In further such embodiments wherein R₁ is —C(═O)NR₄Rs and R₂ is NHR₆, R₄ and R₆ are each alkyl.

In some embodiments, R₁ is —C(═O)NR₄R₅ wherein R₄ heterocycloalkylalkyl; and R₂ is NHR₆, where R₆ is alkyl. In some preferred embodiments, said heterocycloalkylalkyl is pyrrolidino-alkyl.

In some embodiments, R₁ is —C(═O)NR₄R₅ wherein R₄ is alkyl, and R₂ is NHR₆ wherein R₆ is alkyl, arylalkyl optionally substituted with up to three halogen atoms, heteroarylalkyl, or N-alkanoylaminoalkyl. In some preferred embodiments, said arylalkyl is phenylalkyl. In further preferred embodiments, said heteroarylalkyl is furanyl-alkyl.

In some embodiments, R₁ is halogen, alkyl, —C(═O)NH₂, or NO₂.

In some embodiments, R₂ is NHR₆ wherein R₆ is alkyl optionally substituted with dialkylamino, aryloxyalkyl, arylalkyl optionally substituted with up to three groups selected from C₁₋₆ alkoxy, halogen and OH, arylsulfonyl optionally substituted with up to three groups selected from CN and alkyl, —C(═O)aryl optionally substituted with up to three groups selected from CN and halogen, —C(═O)alkyl, heterocycloalkyl optionally substituted with up to three alkyl groups, or urea optionally substituted with aryl, said aryl being optionally substituted with up to three halogen atoms.

In further embodiments, R₁ is halogen, alkyl, —C(═O)NH₂, or NO₂; and R₂ is NHR₆ wherein R₆ is alkyl optionally substituted with dialkylamino, aryloxyalkyl, arylalkyl optionally substituted with up to three groups selected from C₁₋₆ alkoxy, halogen and OH, arylsulfonyl optionally substituted with up to three groups selected from CN and alkyl, —C(═O)aryl optionally substituted with up to three groups selected from CN and halogen, —C(═O)alkyl, heterocycloalkyl optionally substituted with up to three alkyl groups, or urea optionally substituted with aryl, said aryl being optionally substituted with up to three halogen atoms.

In some embodiments, R₁ is halogen, and R₆ is alkyl, aryloxyalkyl, or arylalkyl. In some embodiments, said arylalkyloxy is phenoxyalkyl. In some embodiments, said arylalkyl is phenylalkyl.

In some embodiments, R₁ is alkyl, and R₆ is arylsulfonyl optionally substituted with up to three groups selected from CN and alkyl, —C(═O)aryl optionally substituted with up to three groups selected from CN and halogen, urea optionally substituted with aryl, wherein said aryl is optionally substituted with up to three halogen atoms, —C(═O)alkyl, arylalkyl optionally substituted with up to three groups selected from halogen and OH, or alkyl optionally substituted with dialkylamino. In some embodiments, said arylsulfonyl is phenylsulfonyl. In further embodiments, R₁ is —C(═O)NH₂; and R₆ is arylalkyl, preferably phenylalkyl.

In further embodiments, R₁ is NO₂, and R₆ is alkyl, arylalkyl optionally substituted with up to three C₁₋₆ alkoxy groups, or heterocycloalkyl optionally substituted with alkyl. In some embodiments, said heterocycloalk-yl is piperidinyl.

In some embodiments, p is 1.

The present invention further provides methods for alleviating a symptom of a viral infection comprising administering to a patient suffering from said infection a compound of Formula I or Formula II, or a composition comprising a compound of Formula I or Formula II. In some embodiments, the viral infection is HCV.

The present invention further provides methods for alleviating a symptom of SARS comprising administering to a patient suffering therefrom a compound of Formula I or Formula II, or a composition comprising a compound of Formula I or Formula II.

In further embodiments, the present invention provides methods for treating HCV in a patient suffering therefrom, comprising administering to said patient a therapeutically effective amount of a substituted carbazole, or a substituted 1-amino-calbazole, or a substituted 1-amino-carbazole-6-carboxylic acid amide bearing at least one substituent on each of said 1-amino moiety and said carboxylic acid ainide moiety.

In further embodiments, the present invention provides methods for treating SARS in a patient suffering therefiom, comprising administering to said patient a therapeutically effective amount of a substituted carbazole, or a substituted 1-amino-carbazole, or a substituted 1-amino-carbazole-6-carboxylic acid amide bearing at least one substituent on each of said 1-amino moiety and said carboxylic acid amide moiety.

The present invention further provides methods of inhibiting HCV in a patient comprising administering to said patient a therapeutically effective amount of a compound of Formula I or Formula II.

The present invention further provides methods of inhibiting SARS in a patient comprising administering to said patient a therapeutically effective amount of a compound of Formula I or Formula II.

The present invention also provides compounds having the Formula II: 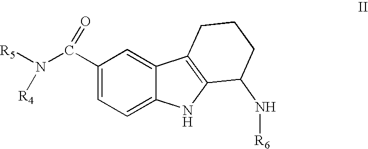
wherein:
R₄ and R₅ are each independently H, alkyl, allyl, alkoxyalkyl, heterocycloalkylalkyl, arylalkyl, heteroarylalkyl, monoalkylaminoalkyl, or dialkylaminoalkyl; wherein said alkyl is optionally substituted with C₁₋₆ alkoxy; and said arylalkyl is optionally substituted with up to three groups selected from dialkylamino, C₁₋₆ alkoxy, perlhaloalkyl and halogen;
or said R₄ and said R₅, together with the nitrogen atom to which they are attached, can form a heterocycloalkyl ring which can optionally be substituted with up to three alkyl groups; and
R₆ is alkyl, heteroarylalkyl, N-alkanoylaminoalkyl, heterocycloalkylalkyl, or arylalkyl optionally substituted with up to three groups selected from halogen and C₁₋₆ alkoxy.

In some embodiments of the compounds of the invention, R₄ is alkyl, heteroarylalkyl, or heterocycloalkylalkyl. In further embodiments of the compounds of the invention, R₆ is alkyl, arylalkyl optionally substituted with up to three groups selected from halogen and C₁₋₆ alkoxy, heteroarylalkyl, and N-alkanoylaminoalkyl.

In further embodiments of the compounds of the invention, R₄ is alkyl, heteroarylalkyl, or heterocycloalkylalkyl, and R₆ is alkyl, arylalkyl optionally substituted with up to three groups selected from halogen and C₁₋₆ alkoxy, heteroarylalkyl, or N-alkanoylaminoalkyl.

In some further embodiments of the compounds of the invention, R₄ is heteroarylalkyl; and R₆ is alkyl or arylalkyl optionally substituted with up to three groups selected from halogen and C₁₋₆ alkoxy. In some embodiments, said arylalkyl is phenylalkyl.

In some further embodiments of the compounds of the invention, R₄ is heterocycloalkylalkyl; and R₆ is alkyl. In some embodiments, said heterocycloalkylalkyl is pynolidino-alkyl.

In some further embodiments of the compounds of the invention, R₄ is alkyl; and R₆ is alkyl, arylalkyl optionally substituted with up to three groups selected from halogen and C₁₋₆ alkoxy, heteroarylalkyl, or N-alkanoylaminoalkyl. In some embodiments, said arylalkyl is phenylalkyl. In further embodiments, said heteroarylalkyl is furanyl-alkyl.

In some embodiments of the compounds of the invention, R₅ is H. In some further embodiments of the compounds of the invention, R₅ is H, and R₄ and R₆ are selected in accordance with Table 1, infra.

Also provided in accordance with the present invention are pharmaceutical compositions comprising at least one compound of the invention.

The present invention also provides methods for alleviating a symptom of a viral infection, and methods for treating a viral infection, comprising administering to a patient suffering from said infection a compound of the invention. In some embodiments of the methods of the invention, the viral infection is HCV or SARS. In further embodiments, the invention provides methods for inhibiting HCV or SARS, comprising administering to a patient suffering therefrom a compound of the invention. In some embodiments of the methods of the invention, the compound of the invention is a substituted carbazole. In further embodiments, the compound is a substituted 1-amino-carbazole-6-carboxylic acid amide bearing at least one substituent on each of said 1-amino moiety and said carboxylic acid amide moiety. In further embodiments of the methods of the invention, the compound has Formula I or Formnula II.

In some embodiments, the present invention provides compounds of Formula I wherein each R₁, R₇ and R₉ are defined as above, and R₂ is —NHR₆, wherein R₆ is cycloalkyl.

In some embodiments, the present invention provides Compounds of Formula II that display IC50 values of less than 10 μM with respect to inhibition HCV as determined by the assay of Example 273 or Example 274, infra.

The present invention also provides compositions containing the subject compounds, and methods for using the subject compounds. Methodologies for making the compounds of the invention are also disclosed. Other useful methodologies will be apparent to those skilled in the art, once armed with the present disclosure. These and other features of the compounds of the subject invention are set forth in more detail below.

### DETAILED DESCRIPTION

In one aspect, the present invention is directed to novel methods and compositions for inhibition of viral infections, particularly HCV and SARS. In some embodiments, the present invention provides methods for alleviating a symptom of a viral infection, and methods for treating a viral infection, comprising administering to a patient suffering from said infection a compound of the invention. In further embodiments, the invention provides methods for inhibiting HCV or SARS, comprising administering to a patient suffering therefrom a compound of the invention. In some embodiments of the methods of the invention, the compound of the invention is a substituted carbazole. In further embodiments, the compound is a substituted 1-amino-carbazole-6-carboxylic acid amide bearing at least one substituent on each of said 1-amino moiety and said carboxylic acid amide moiety. In some embodiments, the substituted carbazole has the Formula I: 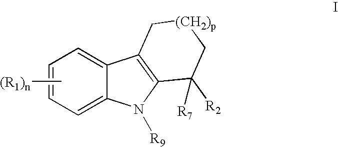
wherein:
each R₁ is independently
a. H, halogen, formyl, carbamoyl, carbamoylamino, carbamoyloxy, NO₂, amino, azido, hydrazino, hydroxylamino, sulfoxyl, sulfonyl, sulfide, disulfide, an ether having 2 to 10 carbon atoms and 1 to 4 oxygen or sulfur atoms;
b. alkyl, alkenyl, alkynyl, perhaloalkyl, alkoxy, alkoxyalkyl, —C(═O)alkyl, —OC(═O)alkyl, —C(═O)alkoxy, alkylsulfonyl, —C(═O)alkylamino, —C(═O)alkylaminoalkyl, —C,(═O)NR₄R₅, —C(═O)NR₄R₆, —NHC(═O)R₇, —C(═O)R₈, monoalkylaminoalkyl, dialkylaminoalkyl, perhaloalkoxy, S-alkyl, urea optionally substituted with aryl wherein said aryl is optionally substituted with up to three halogen atoms;
c. heterocycloalkyl, heterocycloalkylamino, heterocycloalkylnaminoalkyl, heterocycloalkylalkyl, monoalkylaminoalkyl, dialkylaminoalkyl, alkenylaminoalkyl, alkoxyalkylaminoalkyl, heterocycloalkylalkylaminoalkyl,
d. aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl; or
e. heteroaryl, heteroarylalkyl, alkylheteroaryl, heteroarylalkylamino, heteroarylalkylaminoalkyl, arylalkyloxy or arylsulfonyl optionally substituted with up to three groups selected from CN, halogen and alkyl;
wherein any of the foregoing groups can be independently substituted with up to three groups selected from formyl, OH, halogen, C₁₋₆ alkoxy, amino, monoalkylamino, dialkylamino, hydroxyalkyl, arylalkyl, alkyl, aryl, heteroaryl, alkenyl, alkynyl, heteroarylalkyl, CN, perhaloalkyl, monoalkylaminoalkyl, dialkylaminoalkyl, thiol, thioalkoxy, carboxyl, amido, amidino, NO₂, NO₃, perhaloalkoxy, S-alkyl, arylalkyloxy, S-arylalkyl, azido, hydrazino, hydroxylamino, sulfoxyl, sulfonyl, sulfide, disulfide, aryl optionally substituted with up to three halogen atoms, and urea optionally substituted with aryl wherein said aryl is optionally substituted with up to three halogen atoms;
n is 1 to 4;
p is 0 to 2;
R₄ is H, alkyl optionally substituted with C₁₋₆ alkoxy, allyl, alkoxyalkyl, heterocycloalkylalkyl, arylalkyl optionally substituted with up to three groups selected from dialkylamino, C₁₋₆ alkoxy, perhaloalkyl and halogen, heteroarylalkyl, monoalkylaminoalkyl, or dialkylaminoalkyl; wherein said alkyl is optionally substituted with C₁₋₆ alkoxy; and said arylalkyl is optionally substituted with up to three groups selected from dialkylamino, C₁₋₆ alkoxy, perhaloalkyl and halogen;
R₅ is H or alkyl;
or R₄ and R₅, together with the nitrogen atom to which they are attached, can form a heterocycloalkyl ring which can optionally be substituted with up to three alkyl groups;
R₇ and R₈ are independently H, NH₂, alkyl, alkoxy, aryl, heteroaryl, arylalkyl, heteroarylalkyl or heterocycloalkyl, wherein said aryl group can optionally be substituted with up to three groups selected from alkoxy, alkyl, perhaloalkyl, halogen and aryl;
R₂ is heteroaryl, arylalkyl, alkyl, formyl, —C(═O)NH₂, or —NHR₆;
R₆ is H, formyl, alkyl, alkenyl, alkynyl, arylalkyl, heterocycloalkyl, alkylsulfonyl, arylsulfonyl, —C(═O)NH₂, —C(═O)-alkyl, heteroarylalkyl, —C(═O)-alkylaminoalkyl, —C(═O)-aryl, arylalkanoylalkyl, heterocycloalkylalkyl, aryloxyalkyl, monoalkylaminoalkyl, dialkylaminoalkyl, allyl or urea;
wherein:
said alkyl, alkenyl or alkynyl groups can be optionally substituted with up to three groups selected from OH, halogen and C₁₋₆ alkoxy;
said arylalkyl is optionally substituted with up to three groups selected from OH, alkyl, perhaloalkyl, halogen, C₁₋₆ alkoxy, monoalkylamino, dialkylamino and hydroxyalkyl;
said heterocycloalkyl is optionally substituted with up to three groups selected from arylalkyl, alkyl, OH, halogen and C₁₋₆ alkoxy;
said arylsulfonyl is optionally substituted with up to three groups selected from CN, halogen, alkyl, OH, C₁₋₆ alkoxy, monoalkylamino, dialkylamino and hydroxyalkyl;
said —C(═O)-alkyl is optionally substituted with up to three groups selected from OH, halogen, perhaloalkyl and C₁₋₆ alkoxy;
said —C(═O)-aryl is optionally substituted with up to three groups selected from OH, alkyl, perhaloalkyl, halogen, C₁₋6 alkoxy, monoalkylamino, dialkylamino and hydroxyalkyl
said heterocycloalkylalkyl is optionally substituted with up to three groups selected from OH, arylalkyl, alkyl, halogen and C₁₋₆ alkoxy;
said aryloxyalkyl is optionally substituted with up to three groups selected from OH, halogen, C₁₋₆ alkoxy, monoalkylamino, dialkylamino and hydroxyalkyl; and
said urea is optionally substituted with aryl, wherein said aryl is optionally substituted with up to three groups selected from OH, halogen, C₁₋₆ alkoxy, monoalkylamino, dialkylamino and hydroxyalkyl; and
R₉ is H or alkyl.

In some embodiments, the substituted carbazole contains a R₁ group at the 6-position thereof. In some further embodiments, the substituted carbazole contains a single R₁ group. In some further embodiments, the single R₁ group is at the 6-position of the substituted carbazole.

For some embodiments of the invention the term substituted carbazole refers to a compound having a scaffold of the formula: 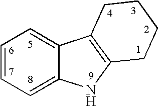
and bearing one or more substituent groups, at one or more of positions 1-9. In other embodiments of the invention the term substituted carbazole refers to scaffolds containing saturated five and seven membered rings bearing one or more substituent groups and having the parent structures below:

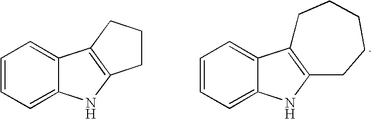

As used herein the term alkyl is intended to mean saturated hydrocarbon species, including straight, branched chain and cyclic hydrocarbons (i.e. “cycloalkyl” groups), for example, methyl, ethyl, n-propyl, isopropyl, n-butyl, sec-butyl, t-butyl, n-pentyl, sec-pentyl, t-pentyl, neopentyl, cyclopropyl, cyclobutyl, cyclopentyl, and cyclohexyl, saturated multiple ring systems such as decahydronaphthalene and adamantane, and the like, including alkyl-substituted derivatives of the foregoing.

A used herein the term alkenyl is intended to denote an alkyl group that contains one or more carbon-carbon double bonds, and is not aromatic. The term alkynyl is intended to denote an alkyl group that contains one or more carbon-carbon triple bonds, and is not aromatic. The term perhaloalkyl is intended to denote an alkyl group in which all hydrogen atoms have been replaced with halogen atoms.

As used herein, the term alkanoyl is intended to denote a group of formula —C(═O)alkyl.

As used herein, the term alkoxy is intended to denote a moiety of formula —O-alkyl. The term perhaloalkoxy is intended to denote an alkoxy group in which all hydrogen atoms have been replaced with halogen atoms. The term “alkoxyalkyl” is intended to denote a group of formula -alkyl-O-alkyl. The terms monoalkylamino and dialkylamino denote, respectively, groups of formula —NH-alkyl and N(alkyl)₂, where the consitiuent alkyl groups can be the same or different. The term “alkylaminoalkyl is intended to denote a group of formula -alkyl-NR′R” where R′ is alkyl, and R″ is H (i.e., “monoalkylaminoalkyl”) or alkyl (i.e., dialkylaminoalkyl). The term “alkoxyalkylaminoalkyl” denotes an alkylaminoalkyl group wherein one or both of the R′ and R″ alkyl groups are substituted with an alkoxy group.

As used herein the term aryl is intended to mean an aromatic hydrocarbon system for example phenyl, naphthyl, phenanthrenyl, anthracenyl, pyrenyl, and the like. In some embodiments, aryl groups have from 6 to 10 carbon atoms.

The term “arylalkoxy” is intended to mean an alkoxy group that bears an aryl group. The term “aryloxyalkyl” is intended to denote a group of formula -alkyl-O-aryl. The term arylcarbonyl is intended to denote a moiety of formula —C(═O)aryl. The term arylalkanoylalkyl is intended to denote a moiety of formula alkyl-C(═O)-arylalkyl. The term arylalkyloxy denotes a group of formula —O-arylalkyl, for example a benzyloxy group. The term alkylheteroaryl denotes a group of formula -heteroaryl-alkyl, for example a 4-methyl-pyrid-2-yl group.

As used herein, the term arylalkyl (or “aralkyl”) is intended to mean an alkyl group that has an aryl group appended thereto, for example benzyl and naphthylmethyl groups. In some embodiments, arylalkyl groups have from 7 to 11 carbon atoms.

As used herein, the term alkylaryl (or “alkaryl”) is intended to mean an aryl group that has one or more alkyl groups appended thereto, for example a 4-methylphen-1-yl group, or a xylyl group attached through the phenyl ring thereof.

The terms “arylamino”, “aralkylamino” and “alkarylamino” respectively denote an aryl, arylalkyl or alkylaryl group that is attached through an amino group of formula —NR″, wherein R″ is H or alkyl. The terms “arylakylaminoalkyl” and “alkylarylaminoalkyl” denote an alkyl group that bears, respectively, an arylalkylamino group or an alkylarylamino group.

As used herein, the term “heterocycloalkyl” is intended to mean a group that contains a nonaromatic ring which contains one or more ring hetero (i.e., non-carbon) atoms which are preferably O, N or S, and which can also contain one or more appended alkyl groups. Also included in the definition of heterocycloalkyl are moieties that contain exocyclic heteroatoms, for example a cycloalkyl ring having a ring carbon attached to an exocyclic O or S atom through a double bond. Also included in the definition of heterocycloalkyl are moieties that having one or more aromatic rings fused (i.e., having a bond in common with) to the nonaromatic heterocyclic ring, for example phthalimidyl, naphthalimidyl pyromellitic diimidyl, phthalanyl, and benzo derivatives of saturated heterocycles such as indolene and isoindolene groups.

The term “heterocycloalkylamino” denotes a heterocycloalkyl group that is attached through an amino group of formula —NR″, wherein R″ is H or alkyl. The term “heterocycloalkylaminoalkyl” denotes a heterocycloalkylamino group that is attached through an alkyl group. The term “heterocycloalkylalkyl” denotes a heterocycloalkyl group that is attached through an exocyclic alkyl group thereof. The term “heterocycloalkylalkylaminoalkyl” denotes a group of formula -alkyl-NR″-heterocycloalkylalkyl, wherein R″ is H or alkyl.

As used herein, the term “heteroaryl” means an aryl group that contains one or more ring hetero (i.e., non-carbon) atoms, which are preferably O, N or S. In some embodiments, heteroaryl groups are monocyclic or bicyclic, and have up to four ring hetero atoms. Examples of some prefeffed heteroaryl groups include radicals derived from pyrrole, pyrazole, imidazole, triazoles, tetrazole, pyridine, pyrazine, pyridazine, pyrimidine, triazines, quinolines, indoles, benzimidazoles, and the like.

The term “heteroarylcarbonyl” is intended to denote a moiety of formula —C(═O)-heteroaryl. The term “heteroarylalkyl” is intended to denote a group of formula -alkyl-heteroaryl. The tenn “alkylheteroaryl” is intended to denote a group of formula -heteroaryl-alkyl. The term “heteroarylalkylamino” denotes a group of formula —NR″-heteroarylalkyl, wherein R″ is H or alkyl. The term “heteroarylalkylamninoalkyl” denotes a group of formula -alkyl-heteroarylalkylamino.

The term “halogen” is intended to denote a Group VII element, including include fluorine, chlorine, bromine and iodine.

In general, the suffix “sulfonyl” is intended to mean attachment of the group tlhrough a group having the formula —S(═O)₂—. Thus, the term “alkylsulfonyl” is intended to denote a group of formula —SO₂-alkyl, the term arylsulfonyl is intended to mean a moiety of formula —S(═O)₂-aryl, and the term heteroarylsulfonyl is intended to mean a moiety of formula —S(=O)₂-heteroaryl.

In general, a term containing the suffix “oxy” is intended to mean attaclhment of the group through an oxygen atom. For example, the term “aryloxy” is intended to mean an aryl group attached through an oxygen atom, for example phenoxy, and the term “aryalkyloxy” or “arylalkyloxy” denotes a group of formula —O-arylalkyl which is equivalent to aryl-alkyl-O— which is also equivalent to —O-alkyl-aryl.

As used herein, the term aryloxycarbonyl is intended to mean a moiety of formula —C(═O)—O-aryl, for example phenoxycarbonyl.

As used herein, the term alkoxyalkoxyalkyl is intended to mean a moiety of formula -alkyl-O-alkyl-O-alkyl.

As used herein, the term hydroxyalkyl is intended to mean an alkyl group that has a hydrogen atom thereof replaced with OH.

As used herein, the term alkoxycarbonyl is intended to mean a moiety of formula —C(═O)—O-alkyl.

The term “side chain of a naturally occurring alpha amino acid” is intended to mean the side chain of naturally occurring alpha amino acids, with the exception of glycine, that are known to have the formula H₂N—CHR—COOH, where R is the side chain. Examples of such naturally occurring amino acids include the 20 so called “essential” amino acids, for example serine and thi-eonine. Further side chains of naturally occurring alpha amino acids can be found in Biochemistry, 3rd Edition, Matthews, Van Holde, and Ahem, Addison Wesley Longman, San Francisco, Calif., incorporated by reference herein in its entirety.

In some embodiments, the present invention provides compounds having the Formula (II): 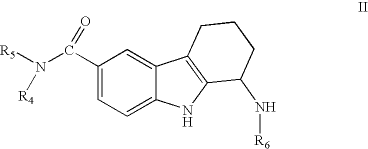
wherein:
R₄ and R₅ are each independently H, alkyl, allyl, alkoxyalkyl, heterocycloalkylalkyl, arylalkyl, heteroarylalkyl, monoalkylaminoalkyl, or dialkylaminoalkyl; wherein said alkyl is optionally substituted with C₁₋₆ alkoxy; and said arylalkyl is optionally substituted with up to three groups selected from dialkylamino, C₁₋₆ alkoxy, perhaloalkyl and halogen;
or said R₄ and said R₅, together with the nitrogen atom to which they are attached, can form a heterocycloalkyl ring which can optionally be substituted with up to three alkyl groups; and
R₆ is alkyl, heteroarylalkyl, N-alkanoylaminoalkyl, heterocycloalkylalkyl, or arylalkyl optionally substituted with up to three groups selected from halogen and C₁₋₆ alkoxy.

In some embodiments of the compounds of the invention, R₄ is alkyl, heteroarylalkyl, or heterocycloalkylalkyl. In further embodiments of the compounds of the invention, R₆ is alkyl, arylalkyl optionally substituted with up to three groups selected from halogen and C₁₋₆ alkoxy, heteroarylalkyl, or N-alkanoylaminoalkyl.

In further embodiments of the compounds of the invention, R₄ is alkyl, heteroarylalkyl, or heterocycloalkylalkyl; and R₆ is alkyl, arylalkyl optionally substituted with up to three groups selected from halogen and C₁₋₆ alkoxy, heteroarylalkyl, or N-alkanoylaminoalkyl.

In some further embodiments of the compounds of the invention, R₄ is heteroarylalkyl; and R₆ is alkyl or arylalkyl optionally substituted with up to three groups selected from halogen and C₁₋₆ alkoxy. In some embodiments, said arylalkyl is phenylalkyl.

In some further embodiments of the compounds of the invention, R₄ is heterocycloalkylalkyl; and R₆ is alkyl. In some embodiments, said heterocycloalkylalkyl is pyrrolidino-alkyl.

In some further embodiments of the compounds of the invention, R₄ is alkyl; and R₆ is alkyl, arylalkyl optionally substituted with up to three groups selected from halogen and C₁₋₆ alkoxy, heteroarylalkyl, or N-alkanoylaminoalkyl. In some embodiments, said arylalkyl is phenylalkyl. In further embodiments, said heteroarylalkyl is furanyl-alkyl.

In some embodiments of the compounds of the invention, R₅ is H. In rther embodiments of the compounds of the invention, R₅ is H, and R₄ and R₆ are selected in accordance with Table 1 below:

TABLE 1

| Com- | | |
|---|---|---|
| pound | R₄ | R₆ |
| 1 | phenylmethyl | cyclohexyl |
| 2 | cyclohexylmethyl | cyclohexyl |
| 3 | cyclohexyl | cyclohexyl |
| 4 | ethyl | cyclohexyl |
| 5 | allyl | cyclohexyl |
| 6 | isopropyl | cyclohexyl |
| 7 | methyl | cyclohexyl |
| 8 | 2-methoxyethyl | cyclohexyl |
| 9 | tetrahydrofuran-2- | cyclohexyl |
| | ylmethyl | |
| 10 | 3-phenylpropyl | cyclohexyl |
| 11 | 2-phenylethyl | cyclohexyl |
| 12 | 2-(4-fluorophenyl)ethyl | cyclohexyl |
| 13 | 4- | cyclohexyl |
| | trifluoromethylphenyl- | |
| | methyl | |
| 14 | 4-methoxyphenylmethyl | cyclohexyl |
| 15 | thien-2-yl-methyl | cyclohexyl |
| 16 | 2-oxopyrrolidin-1- | cyclohexyl |
| | ylpropyl | |
| 17 | pyridin-3-yl-methyl | cyclohexyl |
| 18 | (4- | cyclohexyl |
| | dimethylamino)phenyl- | |
| | methyl | |
| 19 | pyridin-3-yl-methyl | 2-(4- |
| | | fluorophenyl)eth-1-yl |
| 20 | 2-(pyrrolidin-1- | cyclohexyl |
| | yl)ethyl | |
| 21 | ethyl | phenylmethyl |
| 22 | pyridin-3-yl-methyl | butyl-1-yl |
| 23 | pyridin-3-yl-methyl | hexyl-1-yl |
| 24 | pyridin-4-yl-methyl | cyclohexyl |
| 25 | pyridin-3-yl-methyl | 4- |
| | | methylcyclohex-1-yl |
| 26 | pyridin-3-yl-methyl | 2-(4- |
| | | chlorophenyl)eth-1-yl |
| 27 | pyridin-3-yl-methyl | cyclohexyl |
| 28 | ethyl | furan-2-yl-methyl |
| 29 | ethyl | 2-(4- |
| | | chlorophenyl)eth-1-yl |
| 30 | ethyl | 2-(4- |
| | | fluorophenyl)eth-1-yl |
| 31 | ethyl | —CH₂—CH₂—NH—C(═O)CH₃ |
| 32 | ethyl | hex-1-yl |
| 33 | ethyl | 3-phenyl-prop-1-yl |
| 34 | H | 2-phenyl-eth-1-yl |
| 35 | ethyl | 4-phenyl-but-1-yl |
| 36 | ethyl | cyclohexyl |
| 37 | pyridin-3-yl-methyl | cyclohexylmethyl |
| 38 | pyridin-3-yl-methyl | furan-2-yl-methyl |
| 39 | ethyl | phenylmethyl |

Substituted carbazole compounds and compounds of Formulas (I) and (II) may be readily synthesized as shown in Scheme 1, the specifics of which are provided in the Examples section.

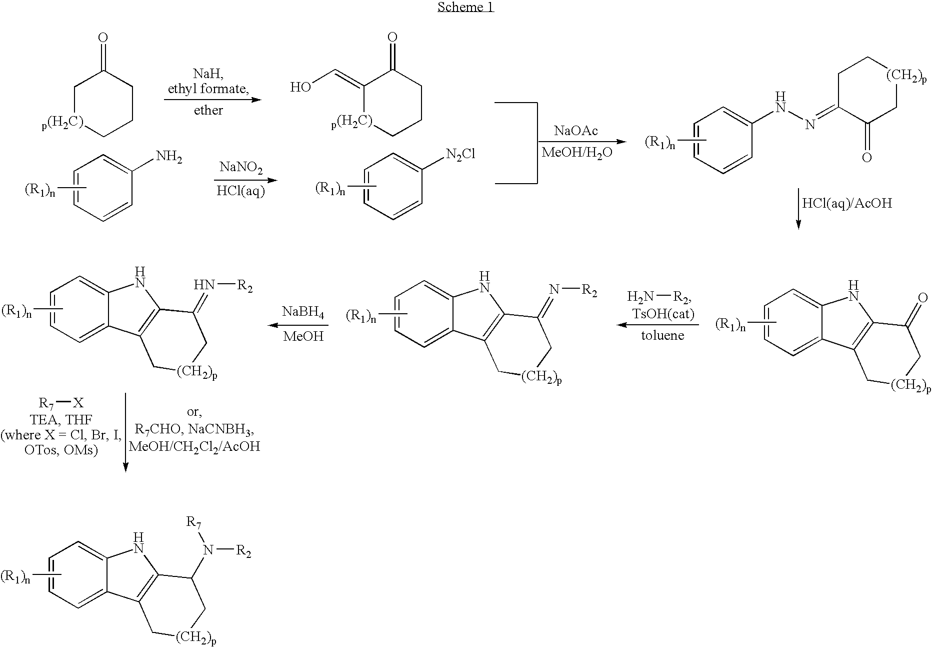

It will be appreciated that by selection of appropriately substituted aniline and cycloalkanone starting materials, a wide variety of substituted carbazole compounds can be prepared, including those of Formulas (I) and (II). Thus, in some embodiments the invention provides for methods of making compounds of Formulas (I) and (II) according to Scheme 1. It is further contemplated that the instant invention covers the intermediates as well as their corresponding methods of synthesis as described in Scheme 1 and the Examples described below. In accordance with such methods, the constituent variables of the compounds can include any of those same values described for the compounds of Formula (I) and (II).

It is contemplated that the present invention include all possible protonated and unprotonated forms of the compounds described herein, as well as solvates and pharmaceutically acceptable salts thereof. It also is intended that each of the compounds described herein specifically include all possible tautomers and stereoisomers.

Throughout the present disclosure, compounds are described by generic and individual chemical formulas, and also by name. In all such instances it is intended that the present invention include each individual stereoisomer of the compounds described herein, as well as racemic forms of the same.

The compounds of the present invention and their pharmaceutically acceptable salts are useful in for the treatment of viral infections in animal and human subjects, in particular HCV and SARS. The compounds of the invention can be used alone, or in a pharmaceutical composition containing one or more compounds of the invention, in combination with one or more pharmaceutically acceptable carriers. Thus, in further aspects, the present invention includes pharmaceutical compositions and methods of treating viral infections utilizing as an active ingredient the novel compounds described herein.

In some embodiments, the compounds of the invention can be prepared as salts, for example and not limitation, amine salts, which can contain any of a variety of pharmaceutically acceptable counterions. Suitable counterions for amine salts include acetate, adipate, aminosalicylate, anhydromethylenecitrate, ascorbate, aspartate, benzoate, benzenesulfonate, bromide, citrate, camphorate, camphorsulfonate, chloride, estolate, ethanesulfonate, fumarate, glucoheptanoate, gluconate, glutamate, lactobionate, malate, maleate, mandelate, methanesulfonate, pantothenate, pectinate, phosphate/diphosphate, polygalacturonate, propionate, salicylate, stearate, succinate, sulfate, tartrate and tosylate. Other suitable anionic species will be apparent to the skilled practitioner.

The compounds of the invention can be formulated in pharmaceutical compositions that can include one or more compounds of the invention and one or more pharmaceutically acceptable carriers. The compounds of the invention can be administered in powder or crystalline form, in liquid solution, or in suspension. They may be administered by a variety of means known to be efficacious for the administration of antiviral agents, including without limitation topically, orally and parenterally by injection (e.g., intravenously or intramuscularly).

When administered by injection, a preferred route of delivery for compounds of the invention is a unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antiviral arts. Another factor influencing the precise dosage regimen, apart from the nature of the infection and peculiar identity of the individual being treated, is the molecular weight of the compound.

The invention described herein also includes a method of treating a viral infection comprising administering to said mammal a compound of the invention in an amount effective to treat said infection. One preferred method of administration of the antiviral compounds of the invention include oral and parenteral, e.g., i.v. infusion, i.v. bolus and i.m. injection.

Compounds provided herein can be formulated into pharmaceutical compositions by admixture with pharmaceutically acceptable nontoxic excipients and carriers. As noted above, such compositions may be prepared for use in parenteral administration, particularly in the form of liquid solutions or suspensions; or oral administration, particularly in the form of tablets or capsules; or intranasally, particularly in the form of powders, nasal drops, or aerosols; or dermally, via, for example, transdermal patches; or prepared in other suitable fashions for these and other forms of administration as will be apparent to those skilled in the art.

The composition may conveniently be administered in unit dosage form and may be prepared by any of the methods well known in the pharmaceutical art, for example, as described in Reminigton's Pharmaceutical Sciences (Mack Pub. Co., Easton, Pa., 1980). Formulations for parenteral administration may contain as common excipients sterile water or saline, polyalkylene glycols such as polyethylene glycol, oils and vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of the active compounds. Other potentially useful parenteral delivery systems for these active compounds include ethylene-vinyl acetate copolymer particles, osmotic pumps, implantable infusion systems, and liposomes. Formulations for inhalation administration contain as excipients, for example, lactose, or may be aqueous solutions containing, for example, polyoxyethylene-9-lauryl ether, glycocholate and deoxycholate, or oily solutions for administration in the form of nasal drops, or as a gel to be applied intranasally. Formulations for parenteral administration may also include glycocholate for buccal administration, a salicylate for rectal administration, or citric acid for vaginal administration. Formulations for transdermal patches are preferably lipoplilic emulsions.

The materials of this invention can be employed as the sole active agent in a pharmaceutical or can be used in combination with other active ingredients, e.g., other agents useful in the treatment of viral infections.

The concentrations of the compounds described herein in a therapeutic composition will vary depending upon a number of factors, including the dosage of the drug to be administered, the chemical characteristics (e.g., hydrophobicity) of the compounds employed, and the route of administration. The compositions for human delivery per unit dosage, whether liquid or solid, may contain from about 0.01% to as high as about 99% of active material, the preferred range being from about 0.1%-60%. For example, the compounds of this invention may be provided in effective inhibitory amounts in an aqueous physiological buffer solution containing about 0.1 to 10% w/v compound for parenteral administration.

Typical dose ranges are from about 1 mg/kg to about 1 g/kg of body weight per day; a preferred dose range is from about 0.01 mg/kg to 100 mg/kg of body weight per day. Such formulations typically provide inhibitory amounts of the compound of the invention. The preferred dosage of drug to be administered is likely, however, to depend on such variables as the type and extent of progression of the disease or disorder, the overall health status of the particular patient, the relative biological efficacy of the compound selected, and formulation of the compound excipient, and its route of administration.

While the present invention has been described with specificity in accordance with certain of its preferred embodiments, the following examples serve only to illustrate the invention and are not intended to limit the same.

Nomenclature for these compounds was provided using ACD Name version 5.04 software (May 28, 2001) available from Advanced Chemistry Development, hic and ChemInnovation NamExpert+Nomenclator™ brand software available from ChemInnovation Software, Inc. Some of the starting materials were named using standard IUPAC nomenclature.

### EXAMPLES

### Example 1

### General Synthesis of Tetrahydrocarbazoles

a) 2-(hydroxymethylene)cyclohexan-1-one

To a suspension of sodium hydride (1.4 eq; 60% dispersion in mineral oil) in dry ethyl ether at 0° C. was added a mixture of cyclohexanone (1.0 eq) and ethyl formate (1.5 eq) over 30 minutes. The reaction was maintained at 0° C. for 5 hours, then allowed to slowly warm to room temperature over 2 hours. After stirring at room temperature for 5 hours, the reaction was quenched with ethanol. The reaction was diluted with ethyl ether and washed with water (3×). The aqueous layers were combined and acidified to pH 5-6 using 6N HCl (aq). The resulting aqueous layer was then extracted with ether (3×). The combined organic layers dried over sodium sulfate. The dry organic filtrate was concentrated in vacuo to yield 2-(hydroxymethylene)cyclohexan-1-one as a crude liquid (LC/MS MH+127.1, Rt 1.68 min).The product oil was used without further purification.

b) Preparation of Hydrazone

To a round bottom flask was added an aniline and concentrated aqueous HCl (1 mL/2.4 mmol of the aniline). Once the mixture was cooled to 20° C. using an ice bath, a solution of sodium nitrite (1 eq) in water was slowly added over 30 min. The reaction was then maintained at 0° C. for 1 hour. A mixture of 2-(hydroxymethylene)cyclohexan-1-one (1.5eq), sodium acetate (2.3 eq) in methanol and water was added to the above diazotized solution over 10 minutes. After stirring at 0° C. for 1 hour, the pure product was filtered from the reaction and washed with water. Vacuum suction was maintained overnight to yield the 2-[aza(phenylamino)methylene]cyclohexan-1-one hydrazone product that was used without further purification.

c) Cyclization to Form Carbazole-1-one.

A solution of substituted 2-[aza(phenylamino)methylene]cyclohexan-1-one, glacial acetic acid and concentrated aqueous HCl (5.8 eq) was heated to reflux for 3 hours. The resulting mixture was allowed to cool to room temperature. The reaction was diluted with water (3 times the reaction volume) and the resulting slurry was extracted with ethyl acetate (4×). The organic layers were combined and dried over sodium sulfate. The resulting filtrate solution was concentrated ill vactio to yield the crude product, which was purified via flash chromatography using a hexanes/ethyl acetate to yield the carbazol-1-one product.

d) Preparation of 1-alkylamino-tetrahydrocarbazole

To a dry round bottom flask was added carbazol-1-one (1 eq), an amine (4 eq), toluene sulfonic acid (catalytic amount), and dry toluene. The reaction was fitted with a Dean-Stark azeotroping apparatus and heated to reflux for 8 hours. Upon cooling to room temperature, the reaction mixture was concentrated in vacuo. Methanol and sodium borohydride (4 eq) were then added to the reaction. Once effervescence ceased, the reaction was fitted with a condenser and heated to reflux for 1 hour. The reaction mixture was concentrated in vactuo, diluted with ethyl acetate, and washed with saturated, aqueous sodium bicarbonate. The organic layer was isolated and the aqueous layer was back extracted with two more portions of ethyl acetate. The organic layers were then combined and dried over sodium sulfate. The filtrate was concentrated to yield crude a product was purified via preparatory HPLC. The pure fractions were combined and lyophilized to yield 1-alkylamino-tetrahydrocarbazole as a TFA salt.

### Example 2

### Preparation of 6-bromo-N-cyclohexyl-2,3,4,9-tetrahydro-1H-carbazol-1-amine

a) 6-bromo-2,3,4,9-tetrahydro-1H-carbazol-1-one

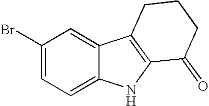

To a round bottom flask was added 4-bromoaniline and concentrated aqueous HCl (5.8 eq). Once the mixture was cooled to 0° C. using an ice bath, a solution of sodium nitrite (1 eq) in water was slowly added over 30 min. The reaction was then maintained at 0° C. for 1 hour. A mixture of 2-(hydroxymethylene)cyclohexan-1-one (1.5 eq), sodium acetate (2.3 eq), methanol, and water was added to the above diazotized solution over 10 minutes. After stirring at 0° C. for 1 hour, the pure product was filtered from the reaction and washed with water. Vacuum suction was maintained overnight to yield the crude intermediate that was then mixed with concentrated aqueous HCl (5.8 eq) in glacial acetic acid and heated to reflux for 3 hours. The resulting mixture was allowed to cool to room temperature. The reaction was diluted with water (3 times the reaction volume) and the resulting slurry was extracted with ethyl acetate (4×). The organic layers were combined and dried over sodium sulfate. The resulting filtrate solution was concentrated in vacuo to yield the crude product, which was purified via flash chromatography using hexanes/ethyl acetate. The pure fractions were combined and concentrated in vacuo to yield 6-bromo-2,3,4,9-tetrahydro-1H-carbazol-1-one as a solid (LC/MS MH+ 264.0, Rₜ 2.81 min).
b) 6-bromo-N-cyclohexyl-2,3,4,9-tetraliydro-1H-carbazol-1-amine

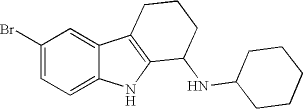

To a dry round bottom flask was added 6-bromo-2,3,4,9-tetrahydro-1H-carbazol-1-one (1 eq), cyclohexylamine (4 eq), toluenesulfonic acid (catalytic amount), and dry toluene (1 mL/0.1 mmol ketone). The reaction was fitted with a Dean-Stark azeotroping apparatus and heated to reflux for 8 hours. Upon cooling to room temperature, the reaction mixture was concentrated in vacuo. Methanol and sodium borohydride (4 eq) were then added to the reaction. Once effervescence ceased, the reaction was fitted with a condenser and heated to reflux for 1 hour. The reaction mixture was concentrated in vacuo, diluted with ethyl acetate, and washed with saturated, aqueous sodium bicarbonate. The organic layer was isolated and the aqueous layer was back extracted with two more portions of ethyl acetate. The organic layers were then combined and dried over sodium sulfate. Once the drying agent was filtered off, the resulting solution was concentrated to yield crude product, which was purified via preparatory HPLC. The pure fractions were combined and lyophilized to yield 6-bromo-N-cyclohexyl-2,3,4,9-tetrahydro-1H-carbazol-1-amine (LC/MS MH+ 345.2, Rₜ 2.74 min) as a TFA salt.

### Example 3

### Preparation of 1-oxo-2,3,4,9-tetrahydro-1H-carbazole-6-carboxylic acid

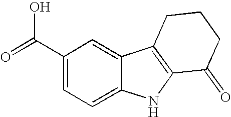

To a round bottom flask was added 4-aminobenzoic acid and concentrated aqueous HCl (5.8 eq). Once the mixture was cooled to 0° C. using an ice bath, a solution of sodium nitrite (1 eq) in water was slowly added over 30 min. The reaction was then maintained at 0° C. for 1 hour. A mixture of 2-(hydroxymethylene)cyclohexan-1-one (1.5 eq), sodium acetate (2.3 eq), methanol, and water was added to the above diazotized solution over 10 minutes. After stirring at 0° C. for 1 hour, the pure product was filtered from the reaction and washed with water. Vacuum suction was maintained overnight to a crude intermediate that was then mixed with glacial acetic acid and concentrated aqueous HCl (5.8 eq) and heated to reflux for 3 hours. The resulting mixture was allowed to cool and sit at room temperature for 3 hours. The fine precipitant was filtered off and washed with water. Vacuum suction was maintained overnight to yield 1-oxo-2,3,4,9-tetrahydro-1H-carbazole-6-carboxylic acid as a solid (LC/MS MH+ 230.3, Rₜ 1.63 min).

### Example 4

### General Procedure for Preparation of 1-alkylamino-6-amido-tetrahydrocarbazole

a) General Procedure for Preparation of 6-amido-1-oxo-tetraliydrocarbazole

A solution of 1-oxo-2,3,4,9-tetrahydro-1H-carbazole-6-carboxylic acid, an amine (RR′NH; 1.5 eq), and EDC (1.1 eq) in dry THF was stirred at room temperature for 8 hours. The reaction was concentrated in vacuo, diluted with ethyl acetate, and washed with water. The organic layer was isolated and the aqueous layer was back extracted with two more portions of ethyl acetate. The organic layers were then combined and dried over sodium sulfate. After filtering off the drying agent, the resulting solution was concentrated in vacuo. The crude was purified via flash chromatography using a methylene chloride/methanol gradient. The pure fractions were concentrated in vacuo to yield 6-amido-1-oxo-tetrahydrocarbazole as a pure solid.

b) General Procedure for Preparation of 1-alkylamino-6-amido-tetrahydrocarbazole

To a dry round bottom flask was added the carbazole ketone (1 eq), an amine (RNH₂; 4 eq), toluenesulfonic acid (catalytic amount), and dry toluene. The reaction was fitted with a Dean-Stark azeotroping apparatus and heated to reflux for 8 hours. Upon cooling to room temperature, the reaction mixture was concentrated in vacuo. Methanol and sodium borohydride (4 eq) were slowly added to the reaction mixture. The reaction was heated to reflux for 1 hour. The reaction mixture was concentrated in vacuo, diluted with ethyl acetate, and washed with saturated, aqueous sodium bicarbonate. The organic layer was isolated and the aqueous layer was back extracted with ethyl acetate (2×). The organic layers were then combined and dried over sodium sulfate. The filtrate was concentrated to yield a residue, which was purified via preparatory HPLC. The pure fractions were combined and lyophilized to yield 1-alkylainino-6-amido-tetrahydrocarbazole as a TFA salt.

### Example 5

### Preparation of N-benzyl-1 -(cyclohexylamino)-2,3,4,9-tetrahydro-1H-carbazole-6-carboxamide

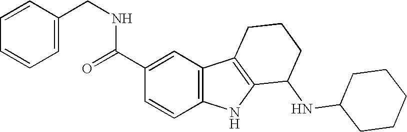

A solution of 1-oxo-2,3,4,9-tetrahydro-1H-carbazole-6-carboxylic acid, benzylamine (1.5 eq), and EDC (1.1 eq) in dry THF was stirred at room temperature for S hours. The reaction was concentrated iil vacuio, diluted with ethyl acetate, and washed with water. The organic layer was isolated and the aqueous layer was back extracted with two more portions of ethyl acetate. The organic layers were then combined and dried over sodium sulfate. After filtering off the drying agent, the resulting solution was concentrated in vacuo. The crude was purified via flash chromatography using a methylene chloride/methanol gradient. The pure fractions were concentrated in vacuo to yield N-benzyl-1-oxo-2,3,4,9-tetrahydro-1H-carbazole-6-carboxamide as a pure solid (LC/MS MH+ 319.2, Rₜ 2.66 min).

To a dry round bottom flask was added N-benzyl-1-oxo-2,3,4,9-tetrahydro-1H-carbazole-6-carboxamide (1 eq), cyclohexylamine (4 eq), toluenesulfonic acid (catalytic amount), and dry toluene. The reaction was fitted with a Dean-Stark azeotroping apparatus and heated to reflux for 8 hours. Upon cooling to room temperature, the reaction mixture was concentrated in vacuo. Methanol and sodium borohydride (4 eq) were slowly added to the reaction mixture. The reaction was heated to reflux for 1 hour. The reaction mixture was concentrated in vacuo, diluted with ethyl acetate, and washed with saturated, aqueous sodium bicarbonate.

The organic layer was isolated and the aqueous layer was back extracted with ethyl acetate (2×). The organic layers were then combined and dried over sodium sulfate. The filtrate was concentrated to yield a residue, which was purified via preparatory HPLC. The pure fractions were combined and lyophilized to yield [8-(cyclohexylamino)(5,6,7,8,9-pentahydro-4aH-carbazol-3-yl)]—N-benzylcarboxamide (LC/MS MH+ 402.4, Rₜ 2.47 min) as a TFA salt.

### Examples 6-272

### Representative Substituted Carbazole Compounds

Representative substituted carbazole compounds of the invention are shown in Table 2. In Table 2, MH+ refers to the molecular ion observed by mass spectrometry.

TABLE 2
Representative substituted carbazoles

| Ex | | | |
|---|---|---|---|
| # | Structure | Name | MH+ |
| 6 | 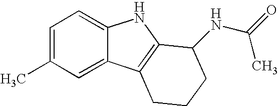 | N-(6-methyl-2,3,4,9- tetrahydro-1H- carbazol-1-yl)acetamide | 243.3 |
| 7 | 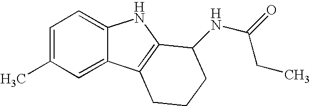 | N-(6-methyl-2,3,4,9- tetrahydro-1H- carbazol-1-yl)propanamide | 257.3 |
| 8 | 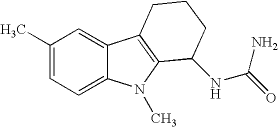 | N-(6,9-dimethyl-2,3,4,9- tetrahydro- 1H-carbazol-1-yl)urea | 258.3 |
| 9 | 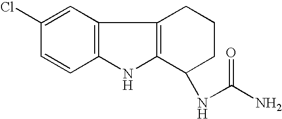 | N-(6-chloro-2,3,4,9- tetrahydro-1H- carbazol-1-yl)urea | 264.7 |
| 10 | 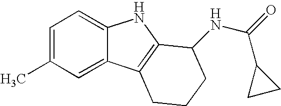 | N-(6-methyl-2,3,4,9- tetrahydro-1H- carbazol-1- yl)cyclopropanecarboxamide | 269.4 |
| 11 | 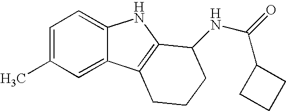 | N-(6-methyl-2,3,4,9- tetrahydro-1H- carbazol-1- yl)cyclobutanecarboxamide | 283.4 |
| 12 | 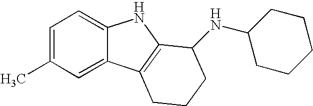 | N-cyclohexyl-6-methyl-2,3,4,9- tetrahydro-1H-carbazol-1-amine | 283.4 |
| 13 | 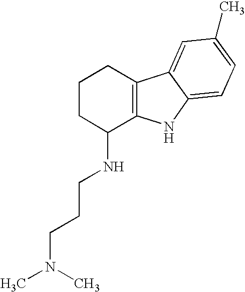 | N,N-dimethyl-N′- (6-methyl-2,3,4,9- tetrahydro-1H-carbazol-1- yl)propane-1,3-diamine | 286.4 |
| 14 | 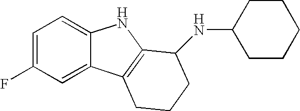 | N-cyclohexyl-6-fluoro-2,3,4,9- tetrahydro-1H-carbazol-1-amine | 287.4 |
| 15 | 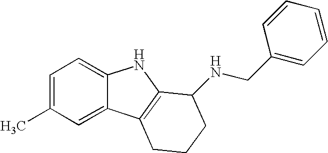 | N-benzyl-6-methyl-2,3,4,9- tetrahydro-1H-carbazol-1-amine | 291.4 |
| 16 | 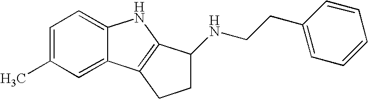 | 7-methyl-N-(2-phenylethyl)- 1,2,3,4- tetrahydrocyclopenta[b]indol-3-amine | 291.4 |
| 17 | 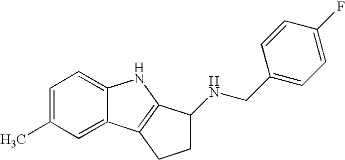 | N-(4-fluorobenzyl)-7-methyl- 1,2,3,4- tetrahydrocyclopenta[b]indol-3-amine | 295.4 |
| 18 | 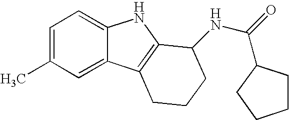 | N-(6-methyl-2,3,4,9- tetrahydro-1H- carbazol-1- yl)cyclopentanecarboxamide | 297.4 |
| 19 | 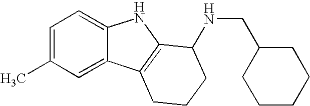 | N-(cyclohexylmethyl)-6-methyl- 2,3,4,9-tetrahydro- 1H-carbazol-1- amine | 297.5 |
| 20 | 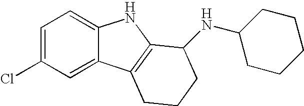 | 6-chloro-N-cyclohexyl-2,3,4,9- tetrahydro-1H-carbazol-1-amine | 303.8 |
| 21 | 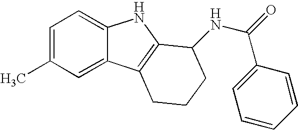 | N-(6-methyl-2,3,4,9- tetrahydro-1H- carbazol-1-yl)benzamide | 305.4 |
| 22 | 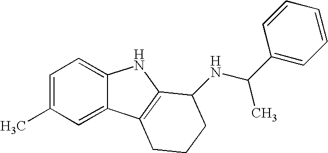 | 6-methyl-N-(1-phenylethyl)- 2,3,4,9- tetrahydro-1H-carbazol-1- amine | 305.4 |
| 23 | 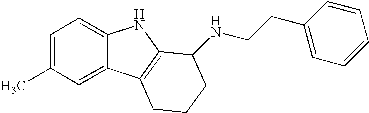 | 6-methyl-N-(2-phenylethyl)- 2,3,4,9- tetrahydro-1H-carbazol-1- amine | 305.4 |
| 24 | 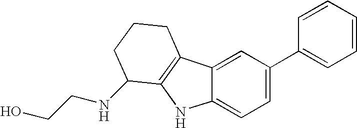 | 2-[(6-phenyl-2,3,4,9- tetrahydro- 1H-carbazol-1-yl)amino]ethanol | 307.4 |
| 25 | 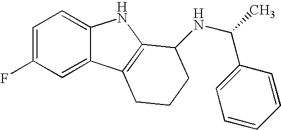 | 6-fluoro-N-[(1R)-1-phenylethyl]- 2,3,4,9- tetrahydro-1H-carbazol-1- amine | 309.4 |
| 26 | 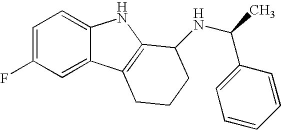 | 6-fluoro-N-[(1S)-1-phenylethyl]- 2,3,4,9- tetrahydro-1H-carbazol-1- amine | 309.4 |
| 27 | 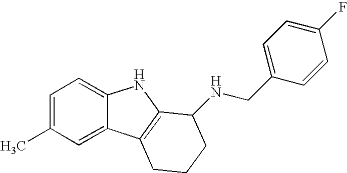 | N-(4-fluorobenzyl)-6-methyl- 2,3,4,9- tetrahydro-1H-carbazol-1- amine | 309.4 |
| 28 | 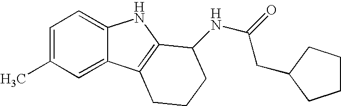 | 2-cyclopentyl-N-(6- methyl-2,3,4,9- tetrahydro-1H-carbazol-1- yl)acetamide | 311.4 |
| 29 | 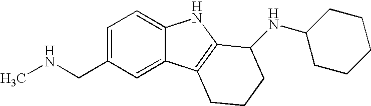 | N-cyclohexyl-6- [(methylamino)methyl]-2,3,4,9- tetrahydro-1H-carbazol-1-amine | 312.5 |
| 30 | 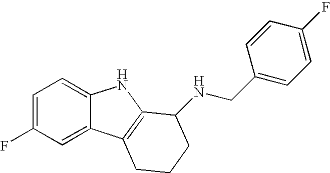 | 6-fluoro-N-(4- fluorobenzyl)-2,3,4,9- tetrahydro-1H-carbazol-1-amine | 313.4 |
| 31 | 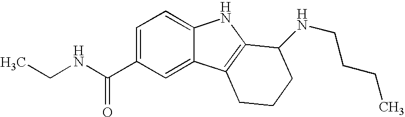 | 1-(butylamino)-N-ethyl-2,3,4,9- tetrahydro-1H-carbazole-6- carboxamide | 314.4 |
| 32 | 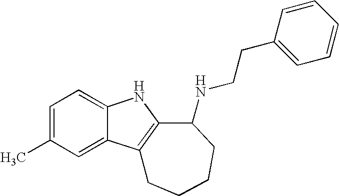 | 2-methyl-N-(2-phenylethyl)- 5,6,7,8,9,10- hexahydrocyclohepta[b]indol-6- amine | 319.5 |
| 33 | 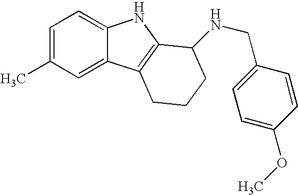 | N-(4-methoxybenzyl)-6-methyl- 2,3,4,9- tetrahydro-1H-carbazol-1- amine | 321.4 |
| 34 | 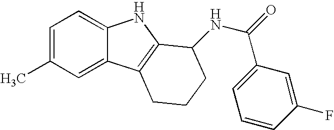 | 3-fluoro-N-(6-methyl-2,3,4,9- tetrahydro-1H-carbazol-1- yl)benzamide | 323.4 |
| 35 | 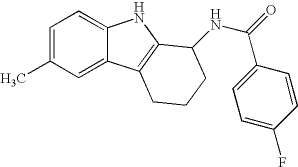 | 4-fluoro-N-(6-methyl-2,3,4,9- tetrahydro-1H-carbazol-1- yl)benzamide | 323.4 |
| 36 | 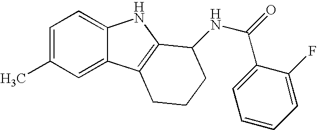 | 2-fluoro-N-(6-methyl-2,3,4,9- tetrahydro-1H-carbazol-1- yl)benzamide | 323.4 |
| 37 | 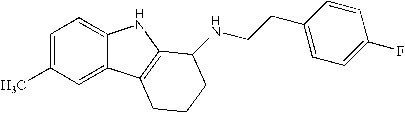 | N-[2-(4-fluorophenyl)ethyl]-6- methyl-2,3,4,9- tetrahydro-1H- carbazol-1-amine | 323.4 |
| 38 | 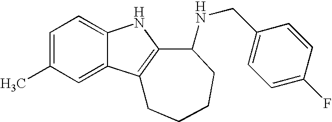 | N-(4-fluorobenzyl)-2-methyl- 5,6,7,8,9,10- hexahydrocyclohepta[b]indol-6-amine | 323.4 |
| 39 | 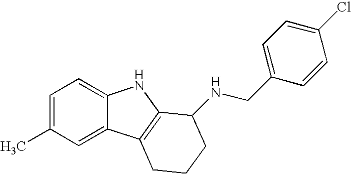 | N-(4-chlorobenzyl)-6-methyl- 2,3,4,9- tetrahydro-1H-carbazol-1- amine | 325.9 |
| 40 | 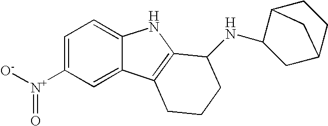 | N-bicyclo[2.2.1]hept-2-yl-6-nitro- 2,3,4,9- tetrahydro-1H-carbazol-1- amine | 326.4 |
| 41 | 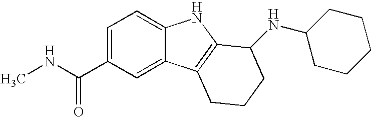 | 1-(cyclohexylamino)-N-methyl- 2,3,4,9- tetrahydro-1H-carbazole-6- carboxamide | 326.5 |
| 42 | 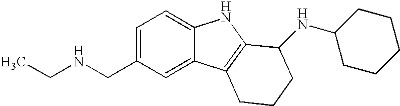 | N-cyclohexyl-6- [(ethylamino)methyl]-2,3,4,9- tetrahydro-1H-carbazol-1-amine | 326.5 |
| 43 | 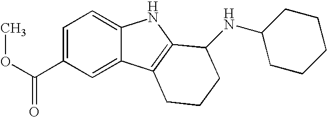 | methyl 1-(cyclohexylamino)- 2,3,4,9- tetrahydro-1H-carbazole-6- carboxylate | 327.4 |
| 44 | 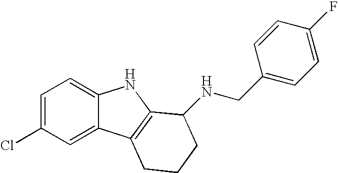 | 6-chloro-N-(4-fluorobenzyl)- 2,3,4,9- tetrahydro-1H-carbazol-1- amine | 329.8 |
| 45 | 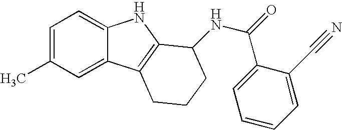 | 2-cyano-N-(6-methyl-2,3,4,9- tetrahydro-1H-carbazol-1- yl)benzamide | 330.4 |
| 46 | 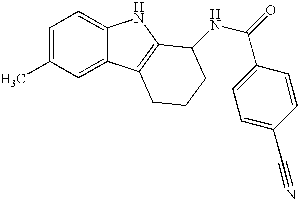 | 4-cyano-N-(6-methyl-2,3,4,9- tetrahydro-1H-carbazol-1- yl)benzamide | 330.4 |
| 47 | 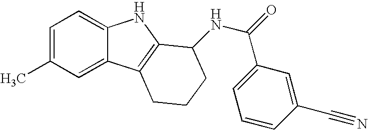 | 3-cyano-N-(6-methyl-2,3,4,9- tetrahydro-1H-carbazol-1- yl)benzamide | 330.4 |
| 48 | 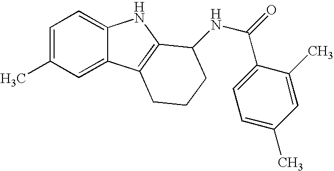 | 2,4-dimethyl-N- (6-methyl-2,3,4,9- tetrahydro-1H-carbazol-1- yl)benzamide | 333.4 |
| 49 | 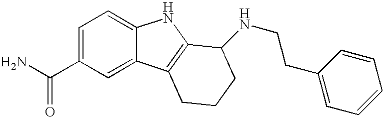 | 1-[(2-phenylethyl) amino]-2,3,4,9- tetrahydro-1H-carbazole-6- carboxamide | 334.4 |
| 50 | 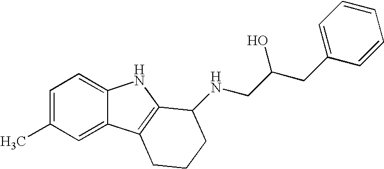 | 1-[(6-methyl-2,3,4,9- tetrahydro- 1H-carbazol-1-yl)amino]-3- phenylpropan-2-ol | 335.5 |
| 51 | 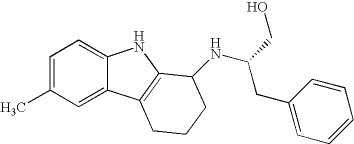 | (2S)-2-[(6-methyl-2,3,4,9- tetrahydro-1H-carbazol-1- yl)amino]-3-phenylpropan-1-ol | 335.5 |
| 52 | 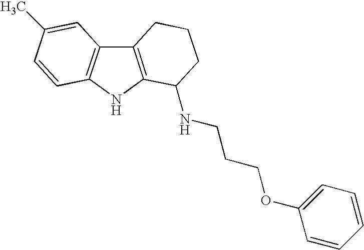 | 6-methyl-N-(3-phenoxypropyl)- 2,3,4,9- tetrahydro-1H-carbazol-1- amine | 335.5 |
| 53 | 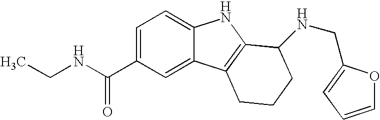 | N-ethyl-1-[(2- furylmethyl)amino]- ′2,3,4,9- tetrahydro-1H-carbazole-6- carboxamide | 338.4 |
| 54 | 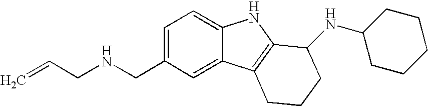 | 6-[(allylamino)methyl]-N- cyclohexyl-2,3,4,9- tetrahydro-1H- carbazol-1-amine | 338.5 |
| 55 | 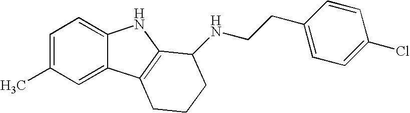 | N-[2-(4-chlorophenyl)ethyl]-6- methyl-2,3,4,9- tetrahydro-1H- carbazol-1-amine | 339.9 |
| 56 | 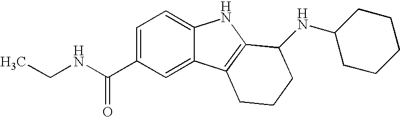 | 1-(cyclohexylamino)-N-ethyl- 2,3,4,9- tetrahydro-1H-carbazole-6- carboxamide | 340.5 |
| 57 | 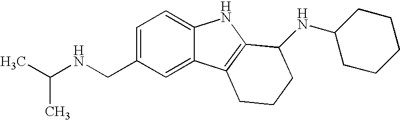 | N-cyclohexyl-6-[(isopropyl- amino)methyl]-2,3,4,9- tetrahydro-1H-carbazol-1-amine | 340.5 |
| 58 | 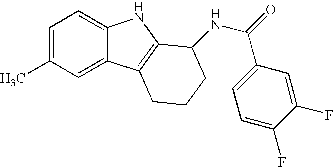 | 3,4-difluoro-N-(6- methyl-2,3,4,9- tetrahydro-1H-carbazol-1- yl)benzamide | 341.4 |
| 59 | 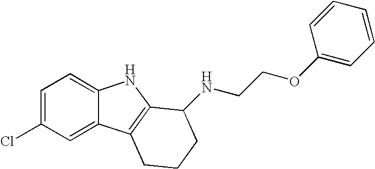 | 6-chloro-N-(2-phenoxyethyl)- 2,3,4,9- tetrahydro-1H-carbazol-1- amine | 341.9 |
| 60 | 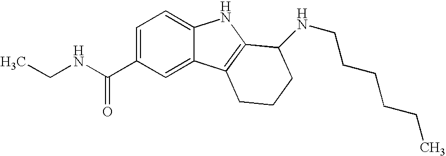 | N-ethyl-1-(hexylamino)-2,3,4,9- tetrahydro-1H-carbazole-6- carboxamide | 342.5 |
| 61 | 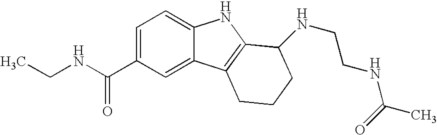 | 1-{[2-(acetylamino) ethyl]amino}-N- ethyl-2,3,4,9- tetrahydro-1H- carbazole-6-carboxamide | 343.4 |
| 62 | 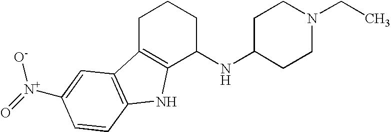 | N-(1-ethylpiperidin- 4-yl)-6-nitro- 2,3,4,9- tetrahydro-1H-carbazol-1- amine | 343.4 |
| 63 | 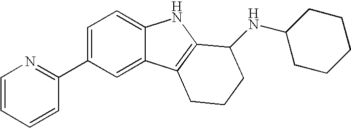 | N-cyclohexyl-6-pyridin-2-yl- 2,3,4,9- tetrahydro-1H-carbazol-1- amine | 346.5 |
| 64 | 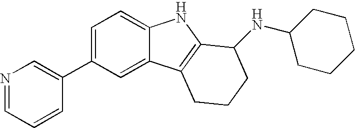 | N-cyclohexyl-6-pyridin-3-yl- 2,3,4,9- tetrahydro-1H-carbazol-1- amine | 346.5 |
| 65 | 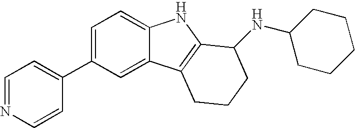 | N-cyclohexyl-6-pyridin-4-yl- 2,3,4,9- tetrahydro-1H-carbazol-1- amine | 346.5 |
| 66 | 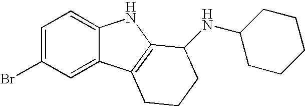 | 6-bromo-N-cyclohexyl-2,3,4,9- tetrahydro-1H-carbazol-1-amine | 348.3 |
| 67 | 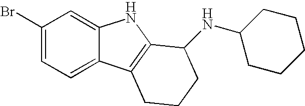 | 7-bromo-N-cyclohexyl-2,3,4,9- tetrahydro-1H-carbazol-1-amine | 348.3 |
| 68 | 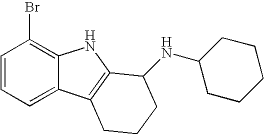 | 8-bromo-N-cyclohexyl-2,3,4,9- tetrahydro-1H-carbazol-1-amine | 348.3 |
| 69 | 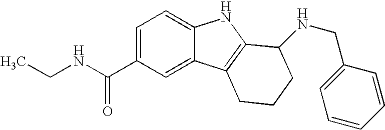 | 1-(benzylamino)- N-ethyl-2,3,4,9- tetrahydro-1H-carbazole-6- carboxamide | 348.5 |
| 70 | 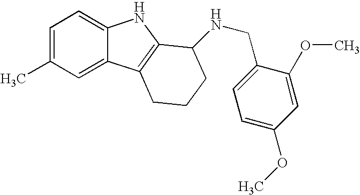 | N-(2,4-dimethoxybenzyl)- 6-methyl- 2,3,4,9- tetrahydro-1H-carbazol-1- amine | 351.5 |
| 71 | 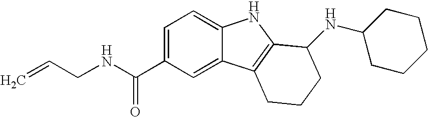 | N-allyl-1-(cyclohexylamino)- 2,3,4,9- tetrahydro-1H-carbazole-6- carboxamide | 352.5 |
| 72 | 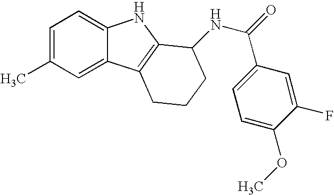 | 3-fluoro-4-methoxy- N-(6-methyl- 2,3,4,9- tetrahydro-1H-carbazol-1- yl)benzamide | 353.4 |
| 73 | 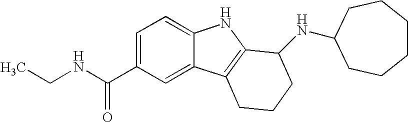 | 1-(cycloheptylamino)-N-ethyl- 2,3,4,9- tetrahydro-1H-carbazole-6- carboxamide | 354.5 |
| 74 | 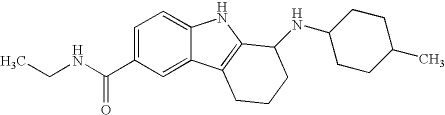 | N-ethyl-1-[(4- methylcyclohexyl) amino]-2,3,4,9- tetrahydro-1H-carbazole-6- carboxamide | 354.5 |
| 75 | 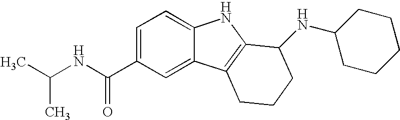 | 1-(cyclohexylamino)- N-isopropyl- 2,3,4,9- tetrahydro-1H-carbazole-6- carboxamide | 354.5 |
| 76 | 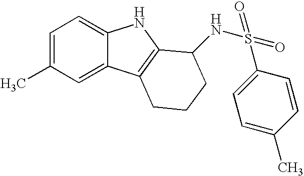 | 4-methyl-N-(6-methyl-2,3,4,9- tetrahydro-1H-carbazol-1- yl)benzenesulfonamide | 355.5 |
| 77 | 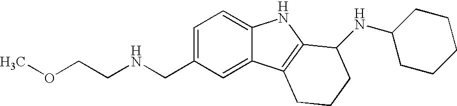 | N-cyclohexyl-6-{[(2- methoxyethyl)amino]methyl}- 2,3,4,9- tetrahydro-1H-carbazol-1- amine | 356.5 |
| 78 | 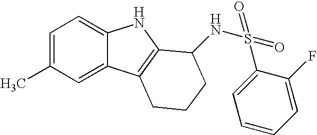 | 2-fluoro-N-(6-methyl-2,3,4,9- tetrahydro-1H-carbazol-1- yl)benzenesulfonamide | 359.4 |
| 79 | 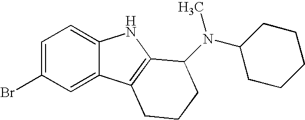 | 6-bromo-N- cyclohexyl-N-methyl- 2,3,4,9- tetrahydro-1H-carbazol-1- amine | 362.3 |
| 80 | 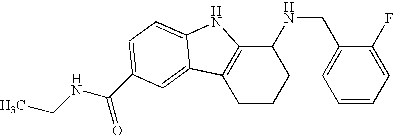 | N-ethyl-1-[(2-fluorobenzyl) amino]- 2,3,4,9- tetrahydro-1H-carbazole-6- carboxamide | 366.4 |
| 81 | 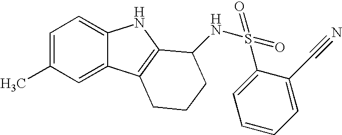 | 2-cyano-N-(6-methyl-2,3,4,9- tetrahydro-1H-carbazol-1- yl)benzenesulfonamide | 366.5 |
| 82 | 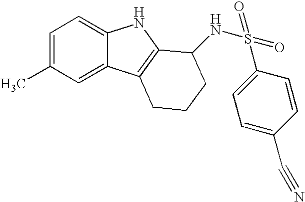 | 4-cyano-N-(6-methyl-2,3,4,9- tetrahydro-1H-carbazol-1- yl)benzenesulfonamide | 366.5 |
| 83 | 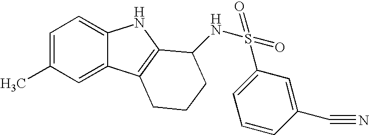 | 3-cyano-N-(6-methyl-2,3,4,9- tetrahydro-1H-carbazol-1- yl)benzenesulfonamide | 366.5 |
| 84 | 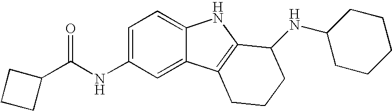 | N-[1-(cyclohexylamino)-2,3,4,9- tetrahydro-1H-carbazol-6- yl]cyclobutanecarboxamide | 366.5 |
| 85 | 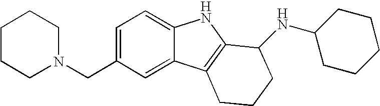 | N-cyclohexyl-6-(piperidin-1- ylmethyl)-2,3,4,9- tetrahydro-1H- carbazol-1-amine | 366.6 |
| 86 | 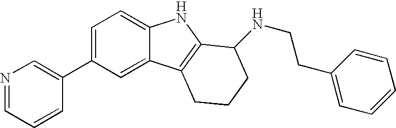 | N-(2-phenylethyl)- 6-pyridin-3-yl- 2,3,4,9- tetrahydro-1H-carbazol-1- amine | 368.5 |
| 87 | 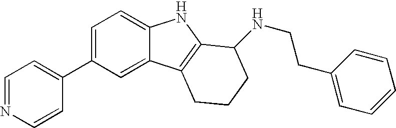 | N-(2-phenylethyl)- 6-pyridin-4-yl- 2,3,4,9- tetrahydro-1H-carbazol-1- amine | 368.5 |
| 88 | 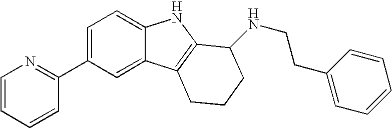 | N-(2-phenylethyl)- 6-pyridin-2-yl- 2,3,4,9- tetrahydro-1H-carbazol-1- amine | 368.5 |
| 89 | 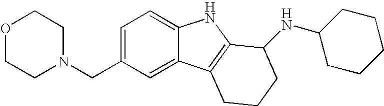 | N-cyclohexyl-6-(morpholin-4- ylmethyl)-2,3,4,9- tetrahydro-1H- carbazol-1-amine | 368.5 |
| 90 | 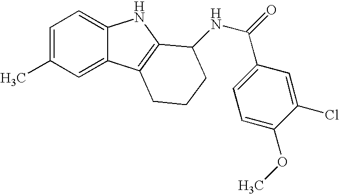 | 3-chloro-4- methoxy-N-(6-methyl- 2,3,4,9- tetrahydro-1H-carbazol-1- yl)benzamide | 369.9 |
| 91 | 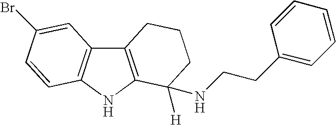 | 6-bromo-N- (2-phenylethyl)-2,3,4,9- tetrahydro-1H-carbazol-1-amine | 370.3 |
| 92 | 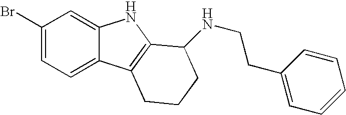 | 7-bromo-N- (2-phenylethyl)-2,3,4,9- tetrahydro-1H-carbazol-1-amine | 370.3 |
| 93 | 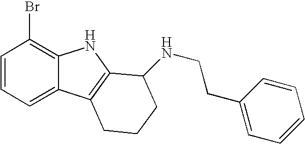 | 8-bromo-N- (2-phenylethyl)-2,3,4,9- tetrahydro-1H-carbazol-1-amine | 370.3 |
| 94 | 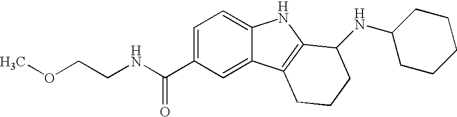 | 1-(cyclohexylamino)-N-(2- methoxyethyl)-2,3,4,9- tetrahydro- 1H-carbazole-6-carboxamide | 370.5 |
| 95 | 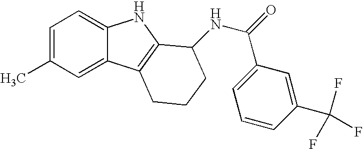 | N-(6-methyl-2,3,4,9- tetrahydro-1H- carbazol-1-yl)-3- (trifluoromethyl)benzamide | 373.4 |
| 96 | 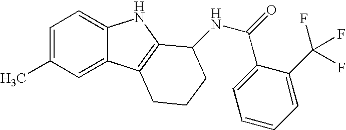 | N-(6-methyl-2,3,4,9- tetrahydro-1H- carbazol-1-yl)-2- (trifluoromethyl)benzamide | 373.4 |
| 97 | 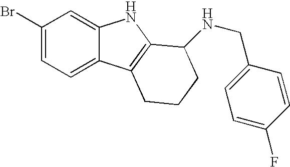 | 7-bromo-N-(4-fluorobenzyl)- 2,3,4,9- tetrahydro-1H-carbazol-1- amine | 374.3 |
| 98 | 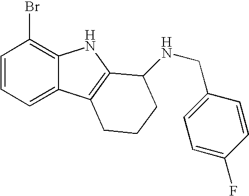 | 8-bromo-N-(4-fluorobenzyl)- 2,3,4,9- tetrahydro-1H-carbazol-1- amine | 374.3 |
| 99 | 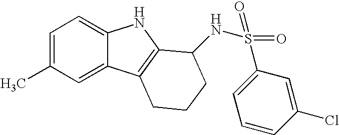 | 3-chloro-N-(6-methyl-2,3,4,9- tetrahydro-1H-carbazol-1- yl)benzenesulfonamide | 375.9 |
| 100 | 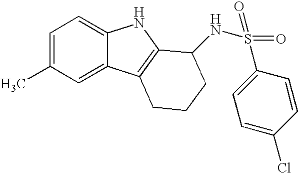 | 4-chloro-N-(6-methyl-2,3,4,9- tetrahydro-1H-carbazol-1- yl)benzenesulfonamide | 375.9 |
| 101 | 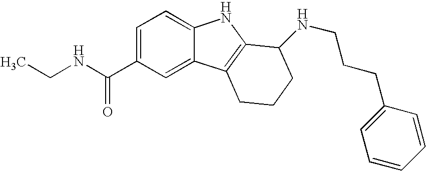 | N-ethyl-1-[(3- phenylpropyl)amino]- 2,3,4,9- tetrahydro-1H-carbazole-6- carboxamide | 376.5 |
| 102 | 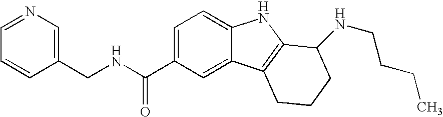 | 1-(butylamino)-N-(pyridin-3- ylmethyl)-2,3,4,9- tetrahydro-1H- carbazole-6-carboxamide | 377.5 |
| 103 | 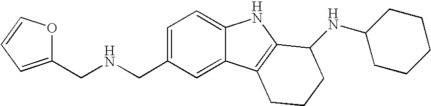 | N-cyclohexyl-6-{[(2- furylmethyl)amino]methyl}-2,3,4,9- tetrahydro-1H-carbazol-1-amine | 378.5 |
| 104 | 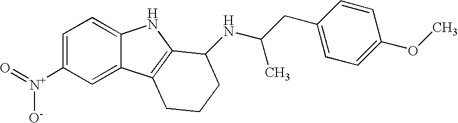 | N-[2-(4-methoxyphenyl)-1- methylethyl]-6-nitro-2,3,4,9- tetrahydro-1H-carbazol-1-amine | 380.5 |
| 105 | 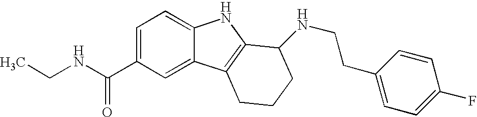 | N-ethyl-1-{[2-(4- fluorophenyl)ethyl]amino}-2,3,4,9- tetrahydro-1H-carbazole-6- carboxamide | 380.5 |
| 106 | 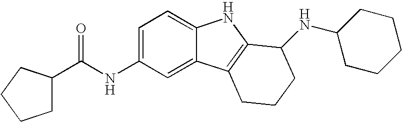 | N-[1-(cyclohexylamino)- 2,3,4,9- tetrahydro-1H-carbazol-6- yl]cyclopentanecarboxamide | 380.5 |
| 107 | 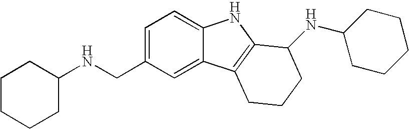 | N-cyclohexyl-6- [(cyclohexylamino) methyl]-2,3,4,9- tetrahydro-1H-carbazol-1-amine | 380.6 |
| 108 | 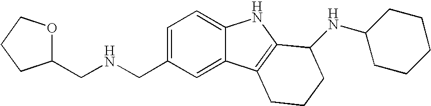 | N-cyclohexyl-6- {[(tetrahydrofuran- 2-ylmethyl)amino]methyl}-2,3,4,9- tetrahydro-1H-carbazol-1-amine | 382.6 |
| 109 | 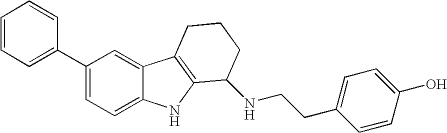 | 4-{2-[(6- phenyl-2,3,4,9- tetrahydro- 1H-carbazol-1- yl)amino]ethyl}phenol | 383.5 |
| 110 | 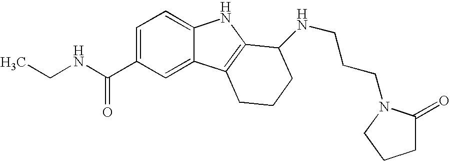 | N-ethyl-1-{[3- (2-oxopyrrolidin-1- yl)propyl]amino}-2,3,4,9- tetrahydro-1H-carbazole-6- carboxamide | 383.5 |
| 111 | 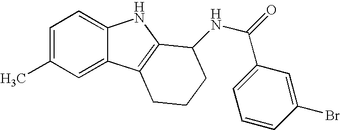 | 3-bromo-N-(6- methyl-2,3,4,9- tetrahydro-1H-carbazol-1- yl)benzamide | 384.3 |
| 112 | 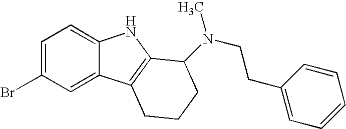 | 6-bromo-N-methyl-N-(2- phenylethyl)-2,3,4,9- tetrahydro- 1H-carbazol-1-amine | 384.3 |
| 113 | 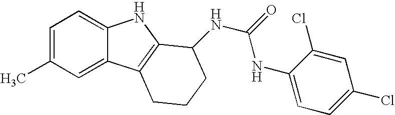 | N-(2,4-dichlorophenyl)-N′-(6- methyl-2,3,4,9- tetrahydro-1H- carbazol-1-yl)urea | 389.3 |
| 114 | 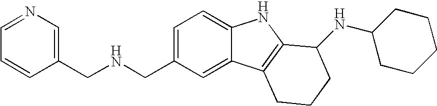 | N-cyclohexyl-6- {[(pyridin-3- ylmethyl)amino]methyl}-2,3,4,9- tetrahydro-1H-carbazol-1-amine | 389.6 |
| 115 | 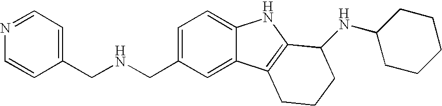 | N-cyclohexyl-6- {[(pyridin-4- ylmethyl)amino]methyl}-2,3,4,9- tetrahydro-1H-carbazol-1-amine | 389.6 |
| 116 | 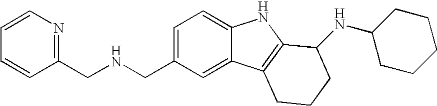 | N-cyclohexyl-6- {[(pyridin-2- ylmethyl)amino]methyl}-2,3,4,9- tetrahydro-1H-carbazol-1-amine | 389.6 |
| 117 | 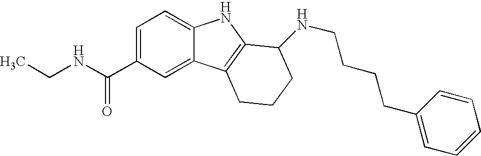 | N-ethyl-1-[(4- phenylbutyl)amino]- 2,3,4,9- tetrahydro-1H-carbazole-6- carboxamide | 390.5 |
| 118 | 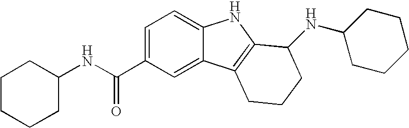 | N-cyclohexyl-1- (cyclohexylamino)- 2,3,4,9- tetrahydro-1H-carbazole-6- carboxamide | 394.6 |
| 119 | 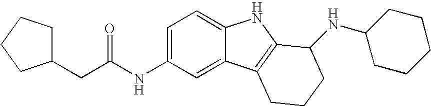 | N-[1-(cyclohexyl- amino)-2,3,4,9- tetrahydro-1H-carbazol-6-yl]-2- cyclopentylacetamide | 394.6 |
| 120 | 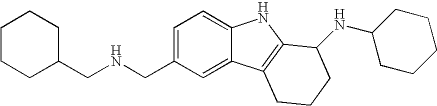 | N-cyclohexyl-6- {[(cyclohexylmethyl) amino]methyl}- 2,3,4,9- tetrahydro-1H-carbazol-1- amine | 394.6 |
| 121 | 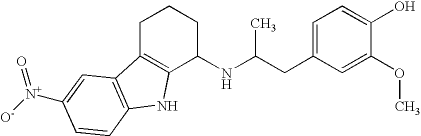 | 2-methoxy-4- {2-[(6-nitro-2,3,4 9- tetrahydro-1H-carbazol-1- yl)amino]propyl}phenol | 396.5 |
| 122 | 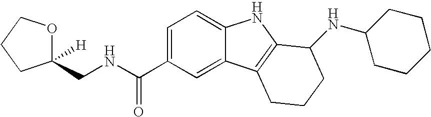 | 1-(cyclohexylamino)-N-[(2R)- tetrahydrofuran-2-ylmethyl]- 2,3,4,9- tetrahydro-1H-carbazole-6- carboxamide | 396.5 |
| 123 | 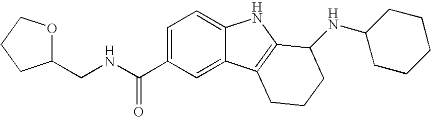 | 1-(cyclohexylamino)-N- (tetrahydrofuran-2-ylmethyl)- 2,3,4,9- tetrahydro-1H-carbazole-6- carboxamide | 396.5 |
| 124 | 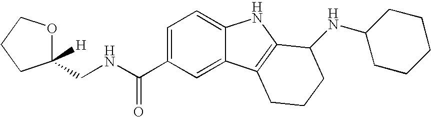 | 1-(cyclohexylamino)-N-[(2S)- tetrahydrofuran-2-ylmethyl]- 2,3,4,9- tetrahydro-1H-carbazole-6- carboxamide | 396.5 |
| 125 | 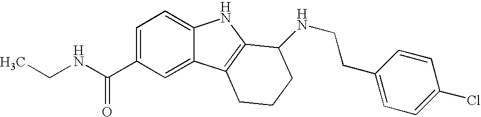 | 1-{[2-(4-chlorophenyl) ethyl]amino}- N-ethyl-2,3,4,9- tetrahydro-1H- carbazole-6-carboxamide | 396.9 |
| 126 | 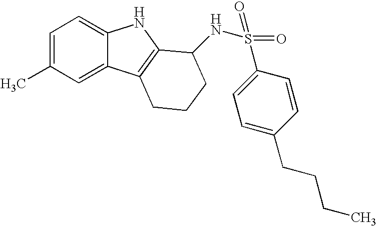 | 4-butyl-N-(6-methyl-2,3,4,9- tetrahydro-1H-carbazol-1- yl)benzenesulfonamide | 397.6 |
| 127 | 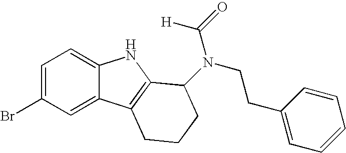 | 6-bromo-2,3,4,9- tetrahydro-1H- carbazol-1-yl(2- phenylethyl)formamide | 398.3 |
| 128 | 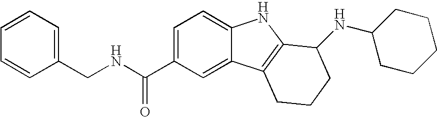 | N-benzyl-1-(cyclohexylamino)- 2,3,4,9- tetrahydro-1H-carbazole-6- carboxamide | 402.6 |
| 129 | 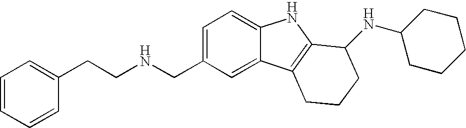 | N-cyclohexyl-6-{[(2- phenylethyl)amino]methyl}-2,3,4,9- tetrahydro-1H-carbazol-1-amine | 402.6 |
| 130 | 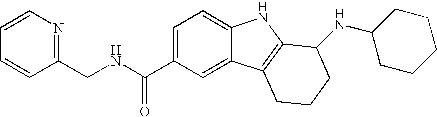 | 1-(cyclohexylamino)-N- (pyridin-2- ylmethyl)-2,3,4,9- tetrahydro-1H- carbazole-6-carboxamide | 403.5 |
| 131 | 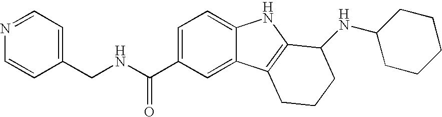 | 1-(cyclohexylamino)-N- (pyridin-4- ylmethyl)-2,3,4,9- tetrahydro-1H- carbazole-6-carboxamide | 403.5 |
| 132 | 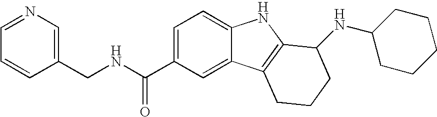 | 1-(cyclohexylamino)-N- (pyridin-3- ylmethyl)-2,3,4,9- tetrahydro-1H- carbazole-6-carboxamide | 403.5 |
| 133 | 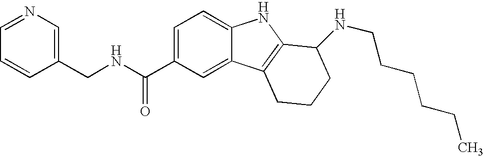 | 1-(hexylamino)-N-(pyridin-3- ylmethyl)-2,3,4,9- tetrahydro-1H- carbazole-6-carboxamide | 405.6 |
| 134 | 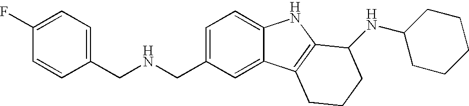 | N-cyclohexyl-6-{[(4- fluorobenzyl)amino]methyl}- 2,3,4,9- tetrahydro-1H-carbazol-1- amine | 406.6 |
| 135 | 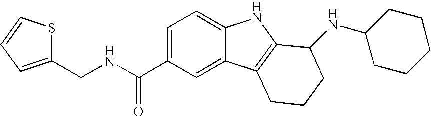 | 1-(cyclohexylamino)-N-(thien-2- ylmethyl)-2,3,4,9- tetrahydro-1H- carbazole-6-carboxamide | 408.6 |
| 136 | 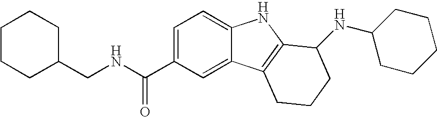 | 1-(cyclohexylamino)-N- (cyclohexylmethyl)-2,3,4,9- tetrahydro-1H-carbazole-6- carboxamide | 408.6 |
| 137 | 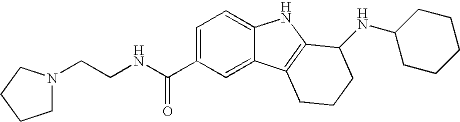 | 1-(cyclohexylamino)-N-(2- pyrrolidin-1-ylethyl)-2,3,4,9- tetrahydro-1H-carbazole-6- carboxamide | 409.6 |
| 138 | 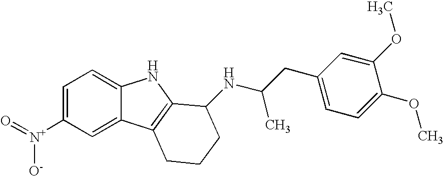 | N-[2-(3,4-dimethoxyphenyl)-1- methylethyl]-6-nitro-2,3,4,9- tetrahydro-1H-carbazol-1-amine | 410.5 |
| 139 | 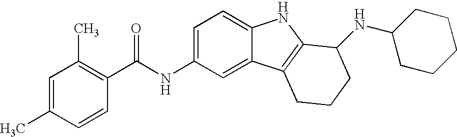 | N-[1-(cyclohexylamino)-2,3,4,9- tetrahydro-1H- carbazol-6-yl]-2,4- dimethylbenzamide | 416.6 |
| 140 | 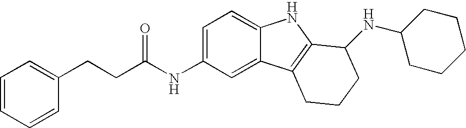 | N-[1-(cyclohexylamino)-2,3,4,9- tetrahydro-1H-carbazol-6-yl]-3- phenylpropanamide | 416.6 |
| 141 | 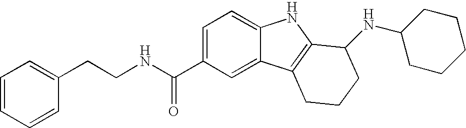 | 1-(cyclohexylamino)-N-(2- phenylethyl)-2,3,4,9- tetrahydro- 1H-carbazole-6-carboxamide | 416.6 |
| 142 | 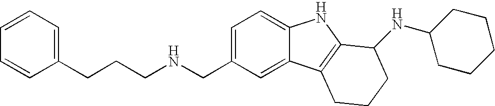 | N-cyclohexyl-6-{[(3- phenylpropyl)amino]methyl}- 2,3,4,9- tetrahydro-1H-carbazol-1- amine | 416.6 |
| 143 | 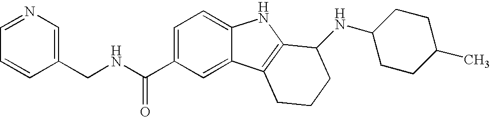 | 1-[(4-methylcyclohexyl) amino]-N- (pyridin-3-ylmethyl)-2,3,4,9- tetrahydro-1H-carbazole-6- carboxamide | 417.6 |
| 144 | 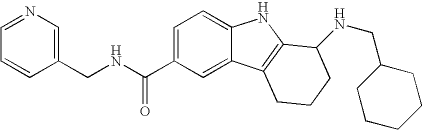 | 1-[(cyclohexylmethyl)amino]-N- (pyridin-3-ylmethyl)-2,3,4,9- tetrahydro-1H-carbazole-6- carboxamide | 417.6 |
| 145 | 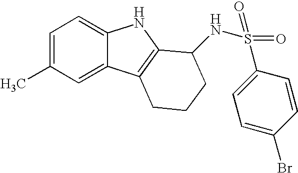 | 4-bromo-N-(6-methyl-2,3,4,9- tetrahydro-1H-carbazol-1- yl)benzenesulfonamide | 420.3 |
| 146 | 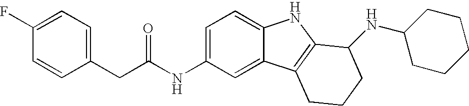 | N-[1- (cyclohexylamino)-2,3,4,9- tetrahydro-1H- carbazol-6-yl]-2-(4- fluorophenyl)acetamide | 420.5 |
| 147 | 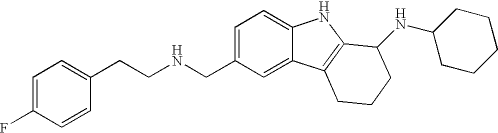 | N-cyclohexyl-6-({[2-(4- fluorophenyl)ethyl]amino}methyl)- 2,3,4,9- tetrahydro-1H-carbazol-1- amine | 420.6 |
| 148 | 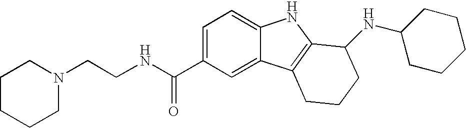 | 1-(cyclohexylamino)-N-(2- piperidin-1-ylethyl)-2,3,4,9- tetrahydro-1H-carbazole-6- carboxamide | 423.6 |
| 149 | 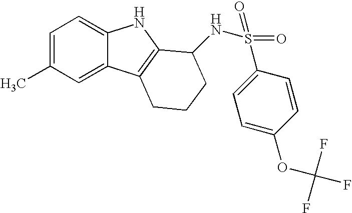 | N-(6-methyl-2,3,4,9- tetrahydro-1H- carbazol-1-yl)-4- (trifluoromethoxy)benzenesul- fonamide | 425.4 |
| 150 | 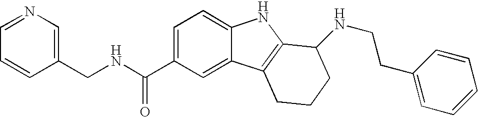 | 1-[(2-phenylethyl)amino]-N- (pyridin-3-ylmethyl)-2,3,4,9- tetrahydro-1H-carbazole-6- carboxamide | 425.5 |
| 151 | 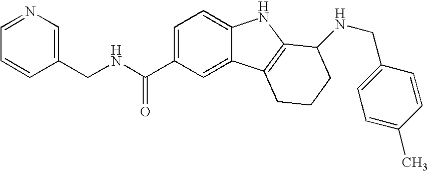 | 1-[(4-methylbenzyl)amino]-N- (pyridin-3-ylmethyl)-2,3,4,9- tetrahydro-1H-carbazole-6- carboxamide | 425.5 |
| 152 | 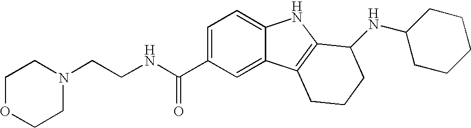 | 1-(cyclohexylamino)-N-(2- morpholin-4-ylethyl)-2,3,4,9- tetrahydro-1H-carbazole-6- carboxamide | 425.6 |
| 153 | 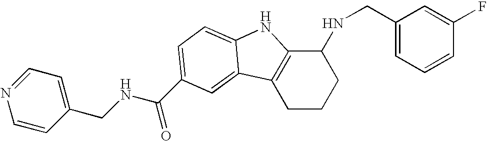 | 1-[(3-fluorobenzyl)amino]-N- (pyridin-4-ylmethyl)-2,3,4,9- tetrahydro-1H-carbazole-6- carboxamide | 429.5 |
| 154 | 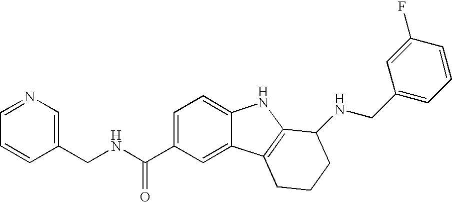 | 1-[(3-fluorobenzyl)amino]-N- (pyridin-3-ylmethyl)-2,3,4,9- tetrahydro-1H-carbazole-6- carboxamide | 429.5 |
| 155 | 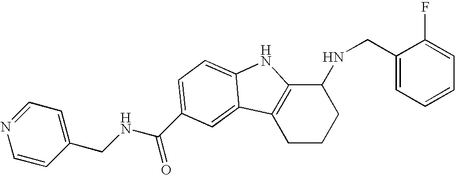 | 1-[(2-fluorobenzyl)amino]-N- (pyridin-4-ylmethyl)-2,3,4,9- tetrahydro-1H-carbazole-6- carboxamide | 429.5 |
| 156 | 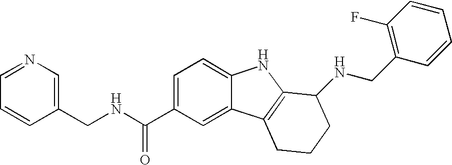 | 1-[(2-fluorobenzyl)amino]-N- (pyridin-3-ylmethyl)-2,3,4,9- tetrahydro-1H-carbazole-6- carboxamide | 429.5 |
| 157 | 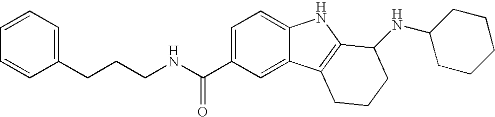 | 1-(cyclohexylamino)-N-(3- phenylpropyl)-2,3,4,9- tetrahydro- 1H-carbazole-6-carboxamide | 430.6 |
| 158 | 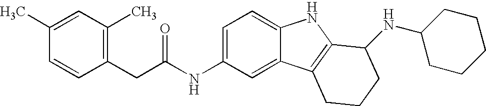 | N-[1-(cyclohexylamino)-2,3,4,9- tetrahydro-1H-carbazol-6-yl]-2- (2,4-dimethylphenyl)acetamide | 430.6 |
| 159 | 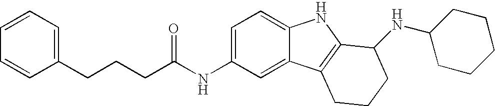 | N-[1-(cyclohexylamino)-2,3,4,9- tetrahydro-1H-carbazol-6-yl]-4- phenylbutanamide | 430.6 |
| 160 | 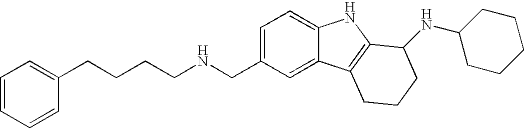 | N-cyclohexyl-6-{[(4- phenylbutyl)amino]methyl}-2,3,4,9- tetrahydro-1H-carbazol-1-amine | 430.6 |
| 161 | 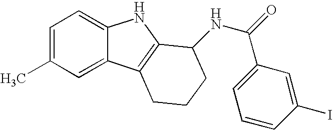 | 3-iodo-N-(6-methyl-2,3,4,9- tetrahydro-1H-carbazol-1- yl)benzamide | 431.3 |
| 162 | 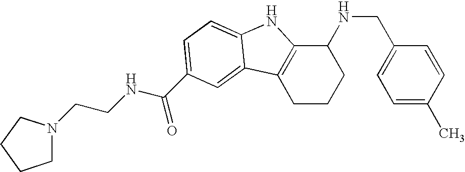 | 1-[(4-methylbenzyl) amino]-N-(2- pyrrolidin-1-ylethyl)-2,3,4,9- tetrahydro-1H-carbazole-6- carboxamide | 431.6 |
| 163 | 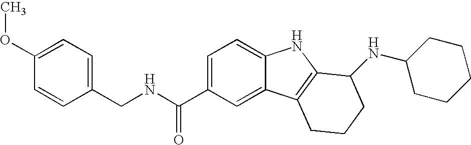 | 1-(cyclohexylamino)-N-(4- methoxybenzyl)-2,3,4,9- tetrahydro-1H-carbazole-6- carboxamide | 432.6 |
| 164 | 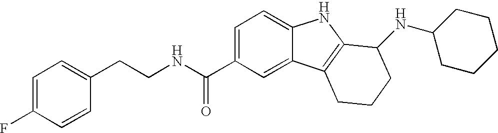 | 1-(cyclohexylamino)-N-[2-(4- fluorophenyl)ethyl]-2,3,4,9- tetrahydro-1H-carbazole-6- carboxamide | 434.6 |
| 165 | 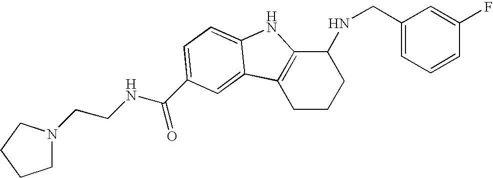 | 1-[(3-fluorobenzyl)amino]-N-(2- pyrrolidin-1-ylethyl)-2,3,4,9- tetrahydro-1H-carbazole-6- carboxamide | 435.6 |
| 166 | 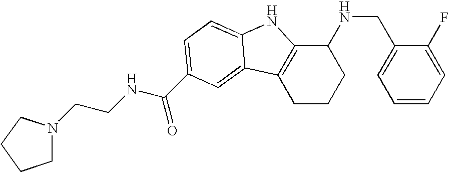 | 1-[(2-fluorobenzyl) amino]-N-(2- pyrrolidin-1-ylethyl)-2,3,4,9- tetrahydro-1H-carbazole-6- carboxamide | 435.6 |
| 167 | 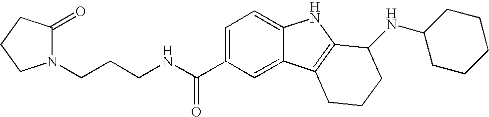 | 1-(cyclohexylamino)-N-[3-(2- oxopyrrolidin-1-yl) propyl]-2,3,4,9- tetrahydro-1H-carbazole-6- carboxamide | 437.6 |
| 168 | 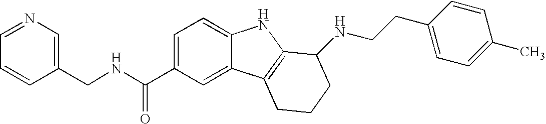 | 1-{[2-(4- methylphenyl)ethyl]amino}-N- (pyridin-3-ylmethyl)-2,3,4,9- tetrahydro-1H-carbazole-6- carboxamide | 439.6 |
| 169 | 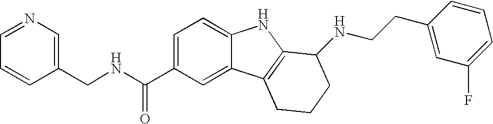 | 1-{[2-(3-fluorophenyl) ethyl]amino}- N-(pyridin-3-ylmethyl)-2,3,4,9- tetrahydro-1H-carbazole-6- carboxamide | 443.5 |
| 170 | 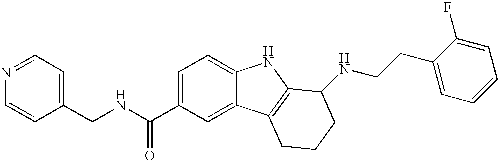 | 1-{[2-(2-fluorophenyl) ethyl]amino}- N-(pyridin-4-ylmethyl)-2,3,4,9- tetrahydro-1H-carbazole-6- carboxamide | 443.5 |
| 171 | 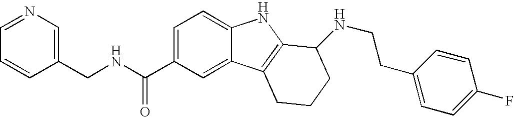 | 1-{[2-(4-fluorophenyl) ethyl]amino}- N-(pyridin-3-ylmethyl)-2,3,4,9- tetrahydro-1H-carbazole-6- carboxamide | 443.5 |
| 172 | 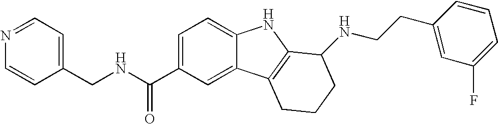 | 1-{[2-(3-fluorophenyl) ethyl]amino}- N-(pyridin-4-ylmethyl)-2,3,4,9- tetrahydro-1H-carbazole-6- carboxamide | 443.5 |
| 173 | 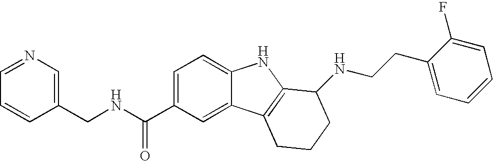 | 1-{[2-(2-fluorophenyl) ethyl]amino}- N-(pyridin-3-ylmethyl)-2,3,4,9- tetrahydro-1H-carbazole-6- carboxamide | 443.5 |
| 174 | 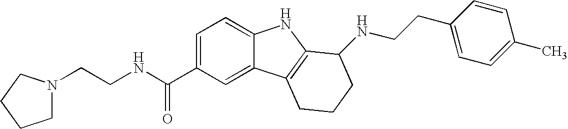 | 1-{[2-(4- methylphenyl)ethyl]amino}-N-(2- pyrrolidin-1-ylethyl)-2,3,4,9- tetrahydro-1H-carbazole-6- carboxamide | 445.6 |
| 175 | 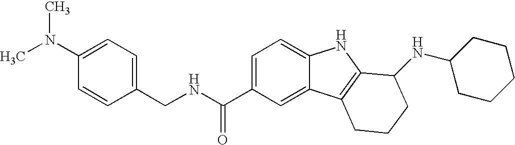 | 1-(cyclohexylamino)-N-[4- (dimethylamino)benzyl]-2,3,4,9- tetrahydro-1H-carbazole-6- carboxamide | 445.6 |
| 176 | 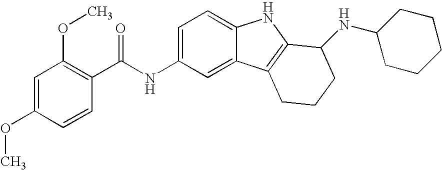 | N-[1-(cyclohexyl- amino)-2,3,4,9- tetrahydro-1H- carbazol-6-yl]-2,4- dimethoxybenzamide | 448.6 |
| 177 | 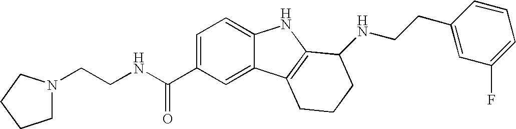 | 1-{[2-(3-fluorophenyl) ethyl]amino}- N-(2-pyrrolidin-1- ylethyl)-2,3,4,9- tetrahydro-1H-carbazole-6- carboxamide | 449.6 |
| 178 | 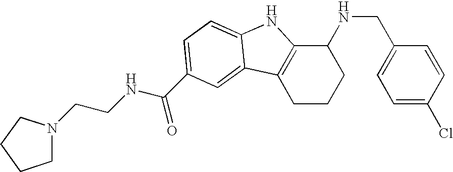 | 1-[(4-chlorobenzyl)amino]-N-(2- pyrrolidin-1-ylethyl)-2,3,4,9- tetrahydro-1H-carbazole-6- carboxamide | 452 |
| 179 | 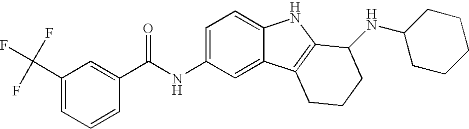 | N-[1-(cyclohexylamino)-2,3,4,9- tetrahydro-1H-carbazol-6-yl]-3- trifluoromethyl)benzamide | 456.5 |
| 180 | 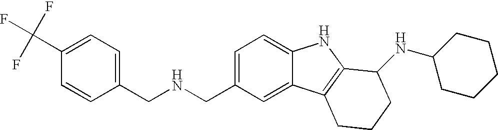 | N-cyclohexyl-6-({[4- trifluoromethyl)benzyl]amino}methyl)-2,3,4,9- tetrahydro-1H- carbazol-1-amine | 456.6 |
| 181 | 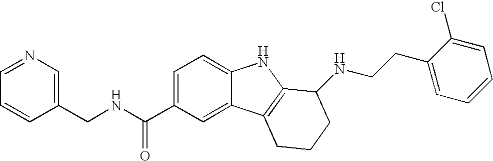 | 1-{[2-(2-chlorophenyl) ethyl]amino}- N-(pyridin-3-ylmethyl)-2,3,4,9- tetrahydro-1H-carbazole-6- carboxamide | 460 |
| 182 | 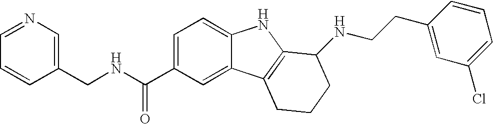 | 1-{[2-(3-chlorophenyl) ethyl[amino}- N-(pyridin-3-ylmethyl)-2,3,4,9- tetrahydro-1H-carbazole-6- carboxamide | 460 |
| 183 | 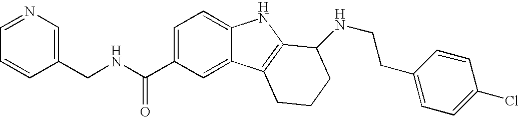 | 1-{[2-(4-chlorophenyl) ethyl]amino}- N-(pyridin-3-ylmethyl)-2,3,4,9- tetrahydro-1H-carbazole-6- carboxamide | 460 |
| 184 | 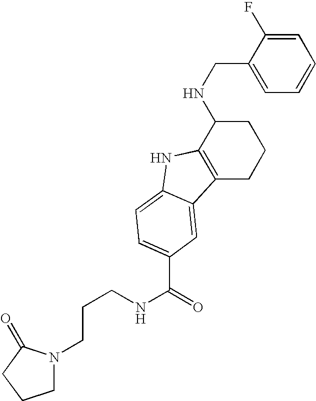 | 1-{[2-fluorobenzyl) amino]-N-[3-(2- oxopyrrolidin-1-yl) propyl]-2,3,4,9- tetrahydro-1H-carbazole-6- carboxamide | 463.6 |
| 185 | 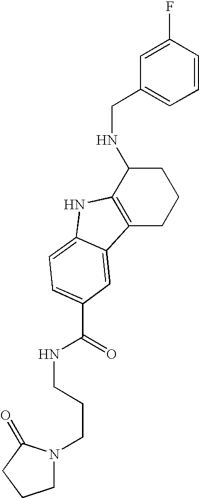 | 1-[(3-fluorobenzyl) amino]-N-[3-(2- oxopyrrolidin-1-yl) propyl]-2,3,4,9- tetrahydro-1H-carbazole-6- carboxamide | 463.6 |
| 186 | 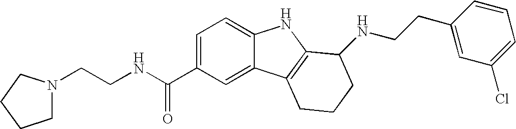 | 1-{[2-(3-chlorophenyl) ethyl]amino}- N-(2-pyrrolidin-1- ylethyl)-2,3,4,9- tetrahydro-1H-carbazole-6- carboxamide | 466 |
| 187 | 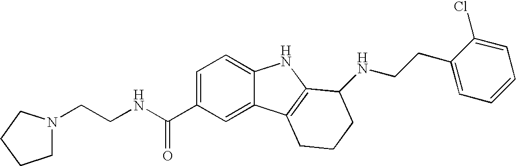 | 1-{[2-(2-chlorophenyl) ethyl]amino}- N-(2-pyrrolidin-1- ylethyl)-2,3,4,9- tetrahydro-1H-carbazole-6- carboxamide | 466 |
| 188 | 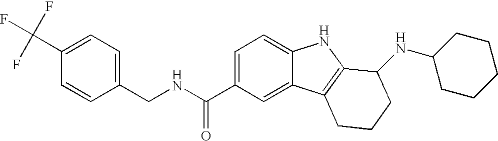 | 1-(cyclohexylamino)-N-[4- trifluoromethyl)benzyl]-2,3,4,9- tetrahydro-1H-carbazole-6- carboxamide | 470.5 |
| 189 | 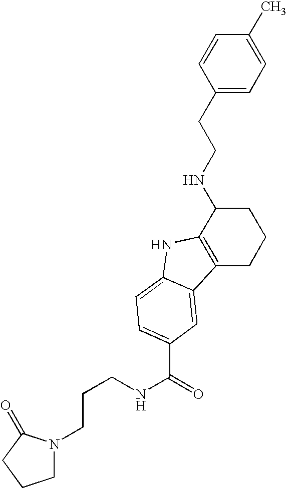 | 1-{[2-(4- methylphenyl)ethyl]amino}-N-[3-(2- oxopyrrolidin-1-yl) propyl]-2,3,4,9- tetrahydro-1H-carbazole-6- carboxamide | 473.6 |
| 190 | 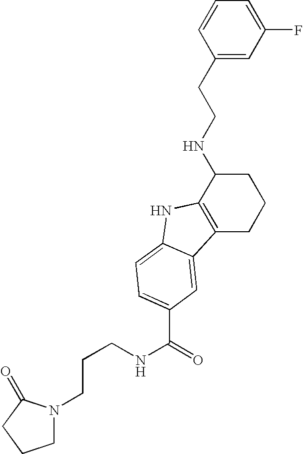 | 1-{[2-(3-fluorophenyl) ethyl]amino}- N-[3-(2-oxopyrrolidin- 1-yl)propyl]- 2,3,4,9- tetrahydro-1H-carbazole-6- carboxamide | 477.6 |
| 191 | 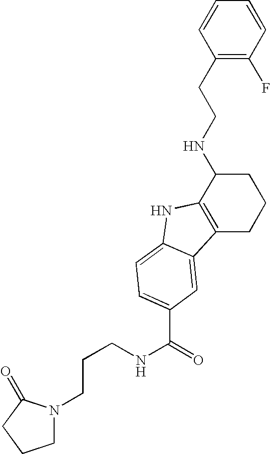 | 1-{[2-(2-fluorophenyl) ethyl]amino}- N-[3-(2-oxopyrrolidin- 1-yl)propyl]- 2,3,4,9- tetrahydro-1H-carbazole-6- carboxamide | 477.6 |
| 192 | 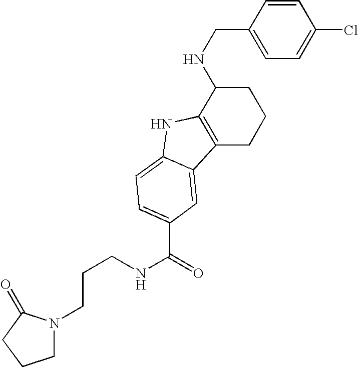 | 1-[(4-chlorobenzyl) amino]-N-[3-(2- oxopyrrolidin-1-yl) propyl]-2,3,4,9- tetrahydro-1H-carbazole-6- carboxamide | 480 |
| 193 | 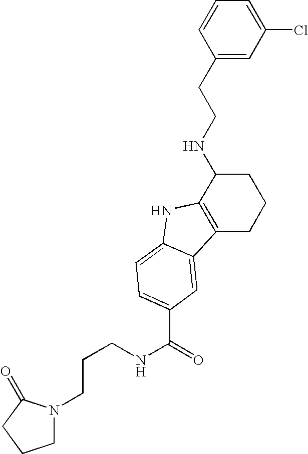 | 1-{[2-(3-chlorophenyl) ethyl]amino}- N-[3-(2-oxopyrrolidin- 1-yl)propyl]- 2,3,4,9- tetrahydro-1H-carbazole-6- carboxamide | 494 |
| 194 | 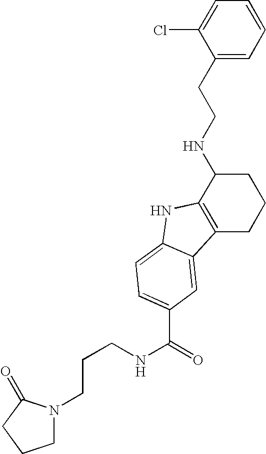 | 1-{[2-(2-chlorophenyl) ethyl]amino}- N-[3-(2-oxopyrrolidin- 1-yl)propyl]- 2,3,4,9- tetrahydro-1H-carbazole-6- carboxamide | 494 |
| 195 | 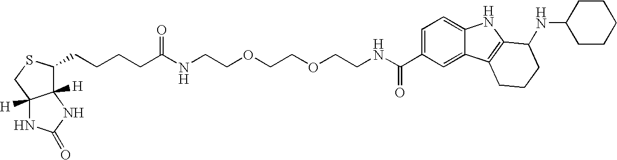 | 1-(cyclohexylamino)-N- (2-{2-[2-({5- [(3aR,4R,6aS)-2-oxohexahydro- 1H-thieno[3,4,-d]imidazol-4- yl]pentanoyl}amino) ethoxy]ethoxy}ethyl)-2,3,4,9- tetrahydro-1H- carbazole-6-carboxamide | 669.9 |
| 196 | 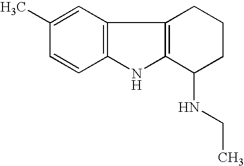 | N-ethyl-6-methyl-2,3,4,9- tetrahydro-1H-carbazol-1-amine | 229.3 |
| 197 | 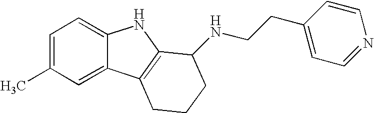 | 6-methyl-N-(2- pyridin-4-ylethyl)- 2,3,4,9- tetrahydro-1H-carbazol-1- amine | 306.4 |
| 198 | 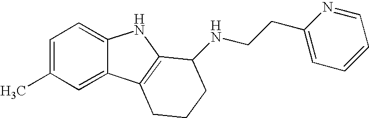 | 6-methyl-N-(2- pyridin-2-ylethyl)- 2,3,4,9- tetrahydro-1H-carbazol-1- amine | 306.4 |
| 199 | 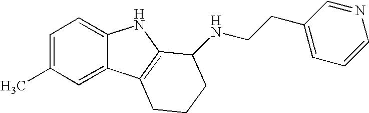 | 6-methyl-N-(2- pyridin-3-ylethyl)- 2,3,4,9- tetrahydro-1H-carbazol-1- amine | 306.4 |
| 200 | 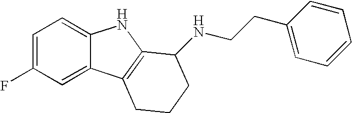 | 6-fluoro-N-(2- phenylethyl)-2,3,4,9- tetrahydro-1H-carbazol-1-amine | 309.4 |
| 201 | 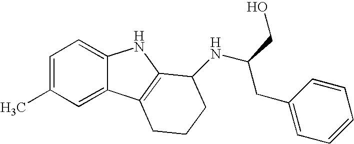 | (2R)-2-[(6-methyl-2,3,4,9- tetrahydro-1H-carbazol-1- yl)amino]-3-phenylpropan-1-ol | 335.5 |
| 202 | 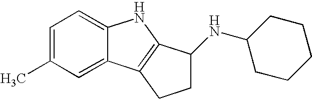 | N-cyclohexyl-7-methyl-1,2,3,4- tetrahydrocyclopenta[b]indol-3- amine | 269.4 |
| 203 | 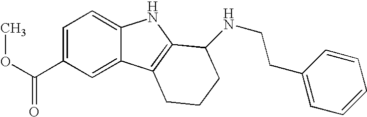 | methyl 1-[(2- phenylethyl)amino]- 2,3,4,9- tetrahydro-1H-carbazole-6- carboxylate | 349.4 |
| 204 | 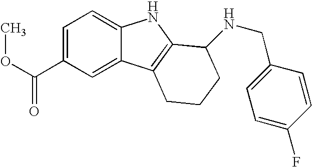 | methyl 1-[(4- fluorobenzyl)amino]- 2,3,4,9- tetrahydro-1H-carbazole-6- carboxylate | 353.4 |
| 205 | 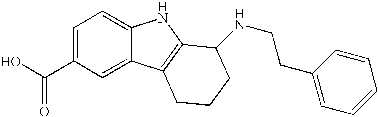 | 1-[(2-phenylethyl) amino]-2,3,4,9- tetrahydro-1H-carbazole-6- carboxylic acid | 335.4 |
| 206 | 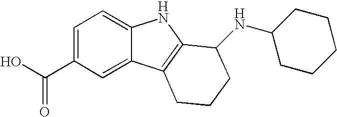 | 1-(cyclohexylamino)- 2,3,4,9- tetrahydro-1H-carbazole-6- carboxylic acid | 313.4 |
| 207 | 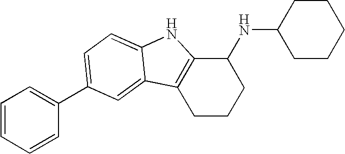 | N-cyclohexyl-6- phenyl-2,3,4,9- tetrahydro-1H-carbazol-1-amine | 345.5 |
| 208 | 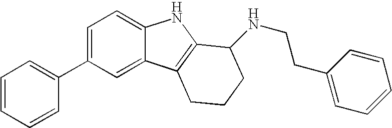 | 6-phenyl-N-(2-phenylethyl)- 2,3,4,9- tetrahydro-1H-carbazol-1- amine | 367.5 |
| 209 | 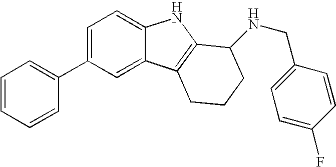 | N-(4-fluorobenzyl)-6-phenyl- 2,3,4,9- tetrahydro-1H-carbazol-1- amine | 371.5 |
| 210 | 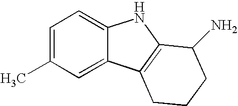 | 6-methyl-2,3,4,9- tetrahydro-1H- carbazol-1-amine | 201.3 |
| 211 | 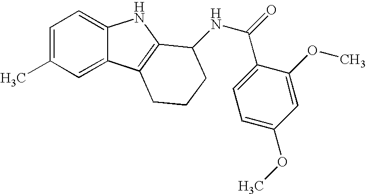 | 2,4-dimethoxy-N-(6-methyl- 2,3,4,9- tetrahydro-1H-carbazol-1- yl)benzamide | 365.4 |
| 212 | 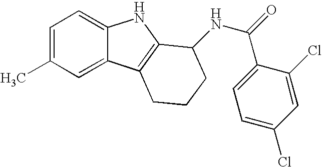 | 2,4-dichloro- N-(6-methyl-2,3,4,9- tetrahydro-1H-carbazol-1- yl)benzamide | 374.3 |
| 213 | 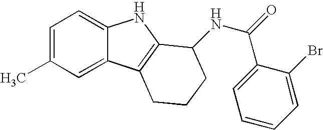 | 2-bromo-N-(6-methyl-2,3,4,9- tetrahydro-1H-carbazol-1- yl)benzamide | 384.3 |
| 214 | 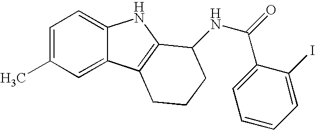 | 2-iodo-N-(6-methyl-2,3,4,9- tetrahydro-1H-carbazol-1- yl)benzamide | 431.3 |
| 215 | 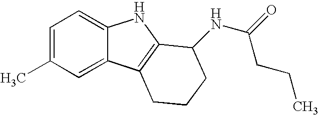 | N-(6-methyl-2,3,4,9- tetrahydro-1H- carbazol-1-yl)butanamide | 271.4 |
| 216 | 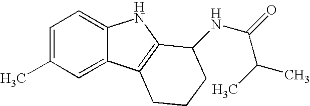 | 2-methyl-N-(6-methyl-2,3,4,9- tetrahydro-1H-carbazol-1- yl)propanamide | 271.4 |
| 217 | 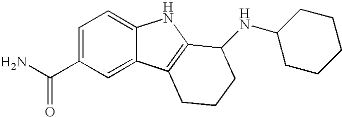 | 1-(cyclohexylamino)-2,3,4,9- tetrahydro-1H-carbazole-6- carboxamide | 312.4 |
| 218 | 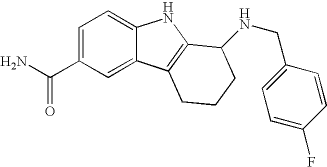 | 1-[(4-fluorobenzyl) amino]-2,3,4,9- tetrahydro-1H-carbazole-6- carboxamide | 338.4 |
| 219 | 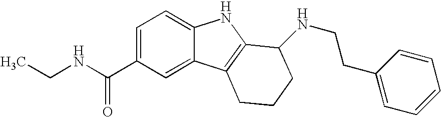 | N-ethyl-1-[(2- phenylethyl)amino]- 2,3,4,9- tetrahydro-1H-carbazole-6- carboxamide | 362.5 |
| 220 | 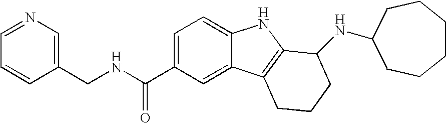 | 1(cycloheptylamino)- N-(pyridin-3- ylmethyl)-2,3,4,9- tetrahydro-1H- carbazole-6-carboxamide | 417.6 |
| 221 | 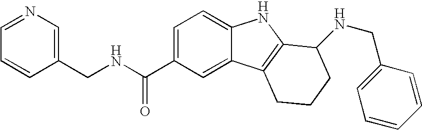 | 1-(benzylamino)-N-(pyridin-3- ylmethyl)-2,3,4,9- tetrahydro-1H- carbazole-6-carboxamide | 411.5 |
| 222 | 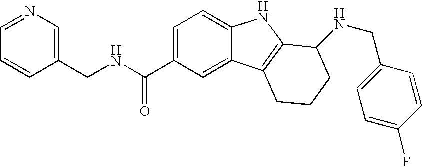 | 1-[(4-fluorobenzyl)amino]-N- pyridin-3-ylmethyl)-2,3,4,9- tetrahydro-1H-carbazole-6- carboxamide | 429.5 |
| 223 | 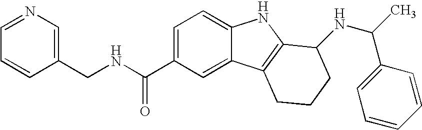 | 1-[(1-phenylethyl)amino]-N- (pyridin-3-ylmethyl)-2,3,4,9- tetrahydro-1H-carbazole-6- carboxamide | 425.5 |
| 224 | 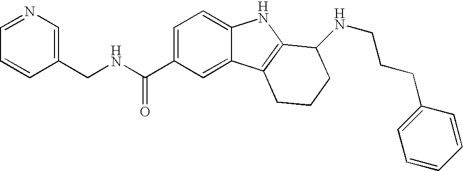 | 1-[(3-phenylpropyl)amino]-N- (pyridin-3-ylmethyl)-2,3,4,9- tetrahydro-1H-carbazole-6- carboxamide | 439.6 |
| 225 | 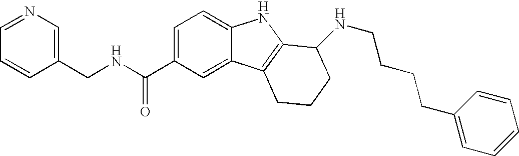 | 1-[(4-phenylbutyl)amino]-N- (pyridin-3-ylmethyl)-2,3,4,9- tetrahydro-1H-carbazole-6- carboxamide | 453.6 |
| 226 | 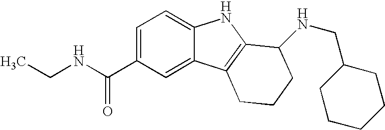 | 1-[(cyclohexylmethyl)amino]-N- ethyl-2,3,4,9- tetrahydro-1H- carbazole-6-carboxamide | 354.5 |
| 227 | 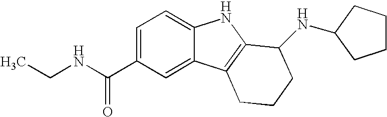 | 1-(cyclopentylamino)-N-ethyl- 2,3,4,9- tetrahydro-1H-carbazole-6- carboxamide | 326.5 |
| 228 | 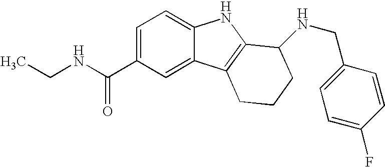 | N-ethyl-1-[(4- fluorobenzyl)amino]- 2,3,4,9- tetrahydro-1H-carbazole-6- carboxamide | 366.4 |
| 229 | 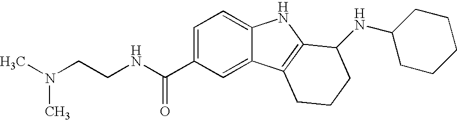 | 1-(cyclohexylamino)-N-[2- (dimethylamino)ethyl]-2,3,4,9- tetrahydro-1H-carbazole-6- carboxamide | 383.5 |
| 230 | 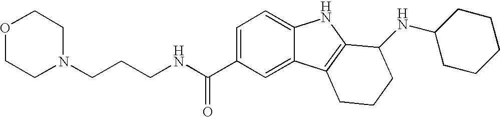 | 1-(cyclohexylamino)-N-(3- morpholin-4-ylpropyl)-2,3,4,9- tetrahydro-1H-carbazole-6- carboxamide | 439.6 |
| 231 | 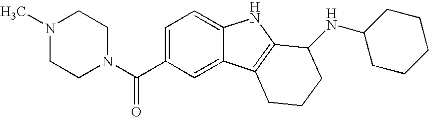 | N-cyclohexyl-6-[(4- methylpiperazin-1-yl)carbonyl]- 2,3,4,9- tetrahydro-1H-carbazol-1- amine | 395.6 |
| 232 | 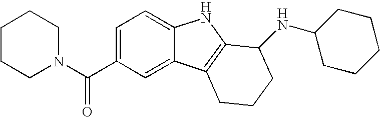 | N-cyclohexyl-6-(piperidin-1- ylcarbonyl)-2,3,4,9- tetrahydro-1H- carbazol-1-amine | 380.5 |
| 233 | 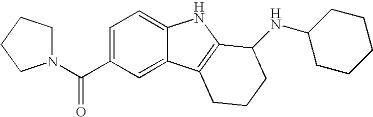 | N-cyclohexyl-6-(pyrrolidin-1- ylcarbonyl)-2,3,4,9- tetrahydro-1H- carbazol-1-amine | 366.5 |
| 234 | 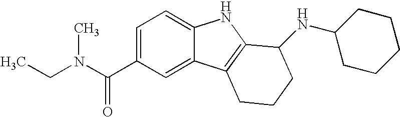 | 1-(cyclohexylamino)-N-ethyl-N- methyl-2,3,4,9- tetrahydro-1H- carbazole-6-carboxamide | 354.5 |
| 235 | 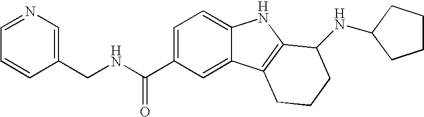 | 1-(cyclopentylamino)- N-(pyridin-3- ylmethyl)-2,3,4,9- tetrahydro-1H- carbazole-6-carboxamide | 389.5 |
| 236 | 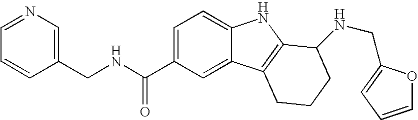 | 1-[(2-furylmethyl)amino]-N- (pyridin-3-ylmethyl)-2,3,4,9- tetrahydro-1H-carbazole-6- carboxamide | 401.5 |
| 237 | 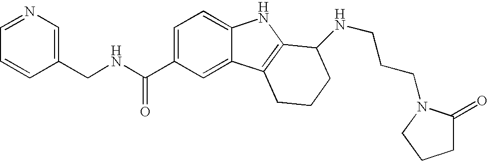 | 1-{[3-(2-oxopyrrolidin-1- yl)propyl[amino}-N-(pyridin-3- ylmethyl)-2,3,4,9- tetrahydro-1H- carbazole-6-carboxamide | 446.6 |
| 238 | 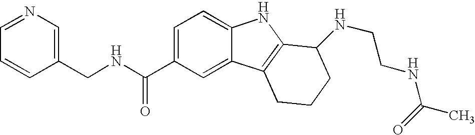 | 1-{[2-(acetylamino) ethyl]amino}-N- (pyridin-3-ylmethyl)-2,3,4,9- tetrahydro-1H-carbazole-6- carboxamide | 406.5 |
| 239 | 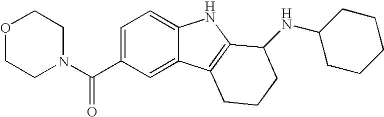 | N-cyclohexyl-6-(morpholin-4- ylcarbonyl)-2,3,4,9- tetrahydro-1H- carbazol-1-amine | 382.5 |
| 240 | 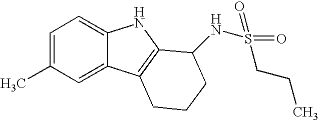 | N-(6-methyl-2,3,4,9- tetrahydro-1H- carbazol-1-yl)propane-1- sulfonamide | 307.4 |
| 241 | 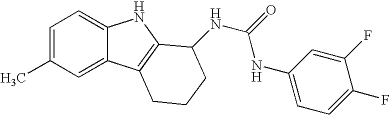 | N-(3,4-difluorophenyl)-N′-(6- methyl-2,3,4,9- tetrahydro-1H- carbazol-1-yl)urea | 356.4 |
| 242 | 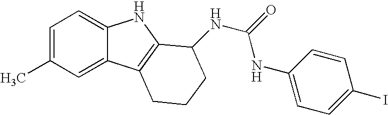 | N-(4-iodophenyl)-N′-(6-methyl- 2,3,4,9- tetrahydro-1H-carbazol-1- yl)urea | 446.3 |
| 243 | 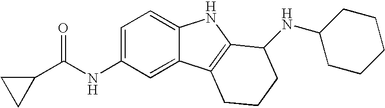 | N-[1-(cyclohexylamino)-2,3,4,9- tetrahydro-1H-carbazol-6- yl]cyclopropanecarboxamide | 352.5 |
| 244 | 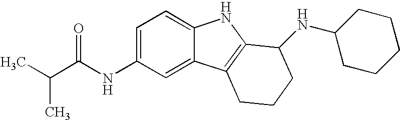 | N-[1-(cyclohexylamino)-2,3,4,9- tetrahydro-1H-carbazol-6-yl]-2- methylpropanamide | 354.5 |
| 245 | 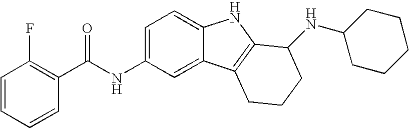 | N-[1-(cyclohexylamino)-2,3,4,9- tetrahydro-1H-carbazol-6-yl]-2- fluorobenzamide | 406.5 |
| 246 | 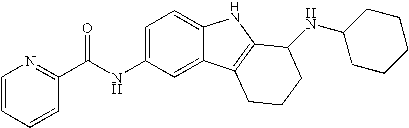 | N-[1-(cyclohexylamino)-2,3,4,9- tetrahydro-1H-carbazol-6- yl]pyridine-2-carboxamide | 389.5 |
| 247 | 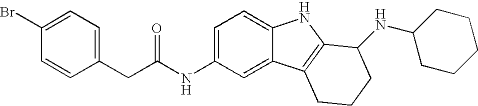 | 2-(4-bromophenyl)-N-[1- (cyclohexylamino)-2,3,4,9- tetrahydro-1H-carbazol-6- yl]acetamide | 481.4 |
| 248 | 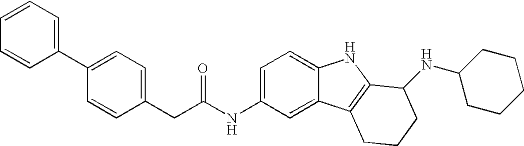 | 2-(1,1′-biphenyl-4-y])-N-[1- (cyclohexylamino)-2,3,4,9- tetrahydro-1H-carbazol-6- yl]acetamide | 478.6 |
| 249 | 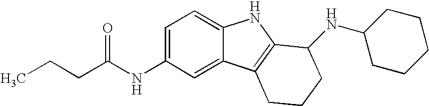 | N-[1-(cyclohexylamino)-2,3,4,9- tetrahydro-1H-carbazol-6- yl]butanamide | 354.5 |
| 250 | 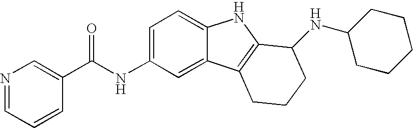 | N-[1-(cyclohexylamino)-2,3,4,9- tetrahydro-1H-carbazol-6- yl]nicotinamide | 389.5 |
| 251 | 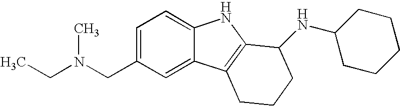 | N-cyclohexyl-6- {[ethyl(methyl)amino]methyl}- 2,3,4,9- tetrahydro-1H-carbazol-1- amine | 340.5 |
| 252 | 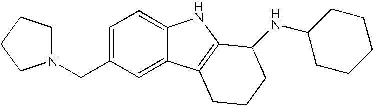 | N-cyclohexyl-6-(pyrrolidin-1- ylmethyl)-2,3,4,9- tetrahydro-1H- carbazol-1-amine | 352.5 |
| 253 | 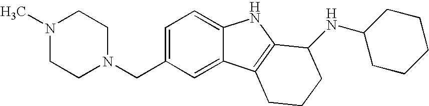 | N-cyclohexyl-6-[(4- methylpiperazin-1-yl)methyl]- 2,3,4,9- tetrahydro-1H-carbazol-1- amine | 381.6 |
| 254 | 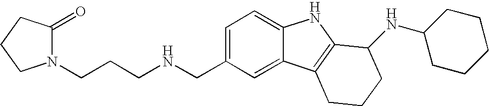 | 1-[3-({[1-(cyclohexylamino)- 2,3,4,9- tetrahydro-1H-carbazol-6- yl]methyl}amino) propyl]pyrrolidin- 2-one | 423.6 |
| 255 | 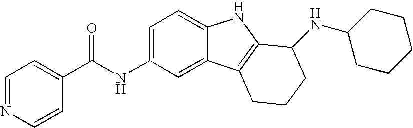 | N-[1-(cyclohexylamino)-2,3,4,9- tetrahydro-1H-carbazol-6- yl]isonicotinamide | 389.5 |
| 256 | 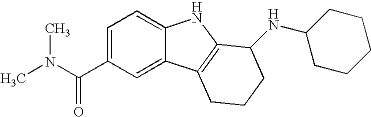 | 1-(cyclohexylamino)- N,N-dimethyl- 2,3,4,9- tetrahydro-1H-carbazole-6- carboxamide | 340.5 |
| 257 | 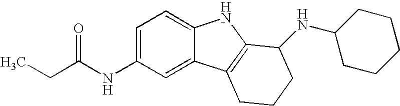 | N-[1-(cyclohexylamino)-2,3,4,9- tetrahydro-1H-carbazol-6- yl]propanamide | 340.5 |
| 258 | 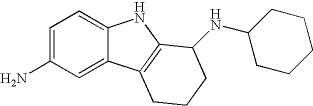 | N˜1˜-cyclohexyl-2,3,4,9- tetrahydro-1H-carbazole-1,6- diamine | 284.4 |
| 259 | 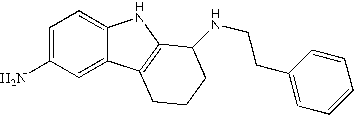 | N˜1˜-(2-phenylethyl)-2,3,4,9- tetrahydro-1H-carbazole-1,6- diamine | 306.4 |
| 260 | 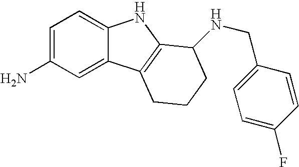 | N˜1˜-(4-fluorobenzyl)-2,3,4,9- tetrahydro-1H-carbazole-1,6- diamine | 310.4 |
| 261 | 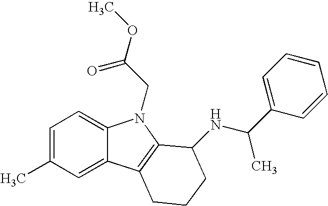 | methyl {6-methyl-1-[(1- phenylethyl)amino]-1,2,3,4- tetrahydro-9H-carbazol-9- yl}acetate | 377.5 |
| 262 | 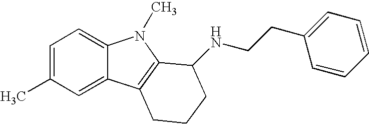 | 6,9-dimethyl-N-(2-phenylethyl)- 2,3,4,9- tetrahydro-1H-carbazol-1- amine | 319.5 |
| 263 | 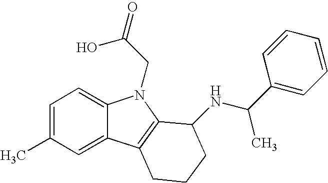 | {6-methyl-1-[(1- phenylethyl)amino]-1,2,3,4- tetrahydro-9H-carbazol- 9-yl}acetic acid | 363.5 |
| 264 | 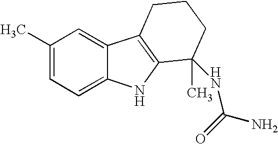 | N-(1,6-dimethyl-2,3,4,9- tetrahydro- 1H-carbazol-1-yl)urea | 258.3 |
| 265 | 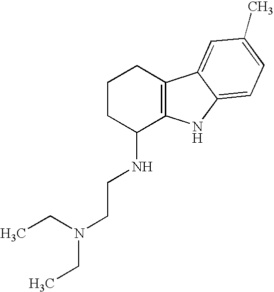 | N,N-diethyl-N′- (6-methyl-2,3,4,9- tetrahydro-1H-carbazol-1- yl)ethane-1,2-diamine | 300.5 |
| 266 | 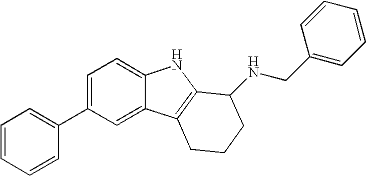 | N-benzyl-6-phenyl-2,3,4,9- tetrahydro-1H-carbazol-1-amine | 353.5 |
| 267 | 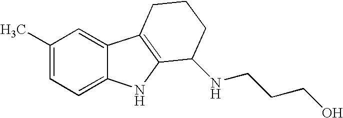 | 3-[(6-methyl-2,3,4,9- tetrahydro- 1H-carbazol-1-yl) amino]propan-1- ol | 259.4 |
| 268 | 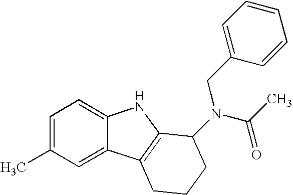 | N-benzyl-N-(6-methyl-2,3,4,9- tetrahydro-1H-carbazol-1- yl)acetamide | 333.4 |

Assay Procedures

### Example 269

### Quantification of HCV Replicon RNA in Cell Lines (HCV Cell Based Assay)

Cell lines, including Huh-11-7 or Huh 9-13, harboring HCV replicons (Lohmalin, et al Science 285:110-113, 1999) are seeded at 5x10³ cells/well in 96 well plates and fed media containing DMEM (high glucose), 10% fetal calf serum, penicillin-streptomycin and non-essential amino acids. Cells are incubated in a 5% CO₂ incubator at 37° C. At the end of the incubation period, total RNA is extracted and purified from cells using Qiagen RNeasy 96 Kit (Catalog No. 74182). To amplify the HCV RNA so that sufficient material can be detected by an HCV specific probe (below), primers specific for HCV (below) mediate both the reverse transcription (RT) of the HCV RNA and the amplification of the cDNA by polymerase chain reaction (PCR) using the TaqMan One-Step RT-PCR Master Mix Kit (Applied Biosystems catalog no. 4309169). The nucleotide sequences of the RT-PCR primers, which are located in the NS5B region of the HCV genome, are the following:

HCV Forward primer “RBNS5bfor”:

5′GCTGCGGCCTGTCGAGCT

HCV Reverse primer “RBNS5Brev”:

5′CAAGGTCGTCTCCGCATAC

Detection of the RT-PCR product was accomplished using the Applied Biosystems (ABI) Prism 7700 Sequence Detection System (SDS) that detects the fluorescence that is emitted when the probe, which is labeled with a fluorescence reporter dye and a quencher dye, is processed during the PCR reaction. The increase in the amount of fluorescence is measured during each cycle of PCR and reflects the increasing amount of RT-PCR product. Specifically, quantification is based on the threshold cycle, where the amplification plot crosses a defined fluorescence threshold. Comparison of the threshold cycles of the sample with a known standard provides a highly sensitive measure of relative template concentration in different samples (ABI User Bulletin #2 Dec. 11, 1997). The data is analyzed using the ABI SDS program version 1.7. The relative template concentration can be converted to RNA copy numbers by employing a standard curve of HCV RNA standards with known copy number (ABI User Bulletin #2 Dec. 11, 1997).

The RT-PCR product was detected using the following labeled probe:

5′FAM-CGAAGCTCCAGGACTGCACGATGCT-TAMRA

FAM=Fluorescence reporter dye.
TAMRA=Quencher dye.

The RT reaction is performed at 48° C. for 30 minutes followed by PCR. Thermal cycler parameters used for the PCR reaction on the ABI Prism 7700 Sequence Detection System were: one cycle at 95° C., 10 minutes followed by 35 cycles each of which included one incubation at 95° C. for 15 seconds and a second incubation for 60° C. for 1 minute.

To normalize the data to an internal control molecule within the cellular RNA, RT-PCR was performed on the cellular messenger RNA glyceraldehydes-3-phosphate dehydrogenase (GAPDH). The GAPDH copy number is very stable in the cell lines used. GAPDH RT-PCR is performed on the same exact RNA sample from which the HCV copy number is determined. The GAPDH primers and probes, as well as the standards with which to determine copy number, is contained in the ABI Pre-Developed TaqMan Assay Kit (catalog no. 4310884E). The ratio of HCV/GAPDH RNA is used to calculate the activity of compounds evaluated for inhibition of HCV RNA replication.

### Example 270

### Activity of Compounds as Inhibitors of HCV Replication (Cell based Assay) in Replicon Containing Huh-7 Cell Lines

The effect of a specific. anti-viral compound on HCV replicon RNA levels in Huh-11-7 or 9-13 cells, cells was determined by comparing the amount of HCV RNA normalized to GAPDH (e.g. the ratio of HCV/GAPDH) in the cells exposed to compound versus cells exposed to the 0% inhibition and the 100% inhibition controls. Specifically, cells were seeded at 5x10³ cells/well in a 96 well plate and were incubated either with: 1) media containing 1% DMSO (0% inhibition control), 2) 100 international units, IU/ml Interferon-alpha 2b in media/1% DMSO or 3) media/1% DMSO containing a fixed concentration of compound. 96 well plates as described above were then incubated at 37° C. for 3 days (primary screening assay) or 4 days (IC50 determination). Percent inhibition was defined as:
% Inhibition=[100−((S−C2)/C1−C2))]×100
wherein:
S=the ratio of HCV RNA copy number/GAPDH RNA copy number in the sample
C1=the ratio of HCV RNA copy iiumber/GAPDH RNA copy number in the 0% inhibition control (media/1% DMSO)
C2=the ratio of HCV RNA copy number/GAPDH RNA copy number in the 100% inhibition control (100 IU/ml Interferon-alpha 2b)

The dose-response curve of the inhibitor was generated by adding compound in serial, three-fold dilutions over three logs to wells starting with the highest concentration of a specific compound at 10 uM and ending with the lowest concentration of 0.01 uM. Further dilution series (1 uM to 0.001 uM for example) was performed if the IC50 value was not in the linear range of the curve. IC50 was determined based on the IDBS Activity Base program using Microsoft Excel “XL Fit” in which A=100% inhibition value (100 IU/ml Interferon-alpha 2b), B=0% inhibition control value (media/1% DMSO) and C=midpoint of the curve as defined as C=(B−A/2)+A. A, B and C values are expressed as the ratio of HCV RNA/GAPDH RNA as determined for each sample in each well of a 96 well plate as described above. For each plate the average of 4 wells were used to define the 100% and 0% inhibition values.

Each of the compounds listed in Table 2, which can be synthesized using the procedures described in Scheme 1 and in Examples 1-5, can be assayed as described above in Example 269 and/or Example 270. Many of these compounds showed activity at less than 10 μGM with respect to inhibition of HCV. More particularly, some compounds of Examples 1-195 showed inhibition of HCV at less than 4 μM. Thus, in some preferred embodiments of the methods and compounds of the invention, the constituent variables of Formulas (I) and (II) are selected from those of Examples 1-195. Additionally, because of the excellent activity of each of these compounds, each of these compounds is individually preferred and is also preferred as a member of a group that includes any or all of the compounds of Examples 1-195, and in the methods described herein. Each of these compounds also are preferred for use in preparation of medicaments for treating biological conditions.

Thus in some embodiments the invention also provides for use of the compounds, stereoisomers, or the pharmaceutically acceptable salts of the present invention in the manufacture of a medicament for the treatment or prophylaxis of a viral infection.

The compounds of Examples 196-268 have not been demonstrated to be effective at a concentration of 10 μM or less using the assay of Example 269 and/or Example 270. However, as compounds that cause HCV inhibition at higher concentrations, such as 10 μM, 20 μM or 50 μM in the assays described herein, can still be useful, the present invention is not intended to be limited to compounds having activity of 10 μM or less. Accordingly, the compounds of Examples 196-268 are also contemplated by the present invention.

It is intended that each of the patents, applications, and printed publications including books mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

What is claimed is:

1. A method for treating a viral infection comprising administering to a patient suffering from said infection a compound, stereoisomer, or phannaceutically acceptable salt of Formula I: 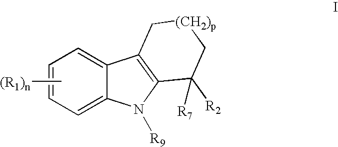
wherein:
each R₁ is independently
a. H, halogen, formyl, carbamoyl, carbamoylamino, carbamoyloxy, NO₂, amino, azido, hydrazino, hydroxylamino, sulfoxyl, sulfonyl, sulfide, disulfide, an ether having 2 to 10 carbon atoms and 1 to 4 oxygen or sulfur atoms;
b. alkyl, alkenyl, alkynyl, perhaloalkyl, alkoxy, alkoxyalkyl, —C(═O)alkyl, —OC(═O)alkyl, —C(═O)alkoxy, alkylsulfonyl, —C(═O)alkylamino, —C(═O)alkylaminoalkyl, —C(═O)N₁R₅, —C(═O)NR₄R₆, —NHC(═O)R₇, —C(═O)R₈, monoalkylaminoalkyl, dialkylaminoalkyl, perhaloalkoxy, S-alkyl, urea optionally substituted with aryl wherein said aryl is optionally substituted with up to three halogen atoms;
c. heterocycloalkyl, heterocycloalkylamino, heterocycloalkylaminoalkyl, heterocycloalkylalkyl, monoalkylaminoalkyl, dialkylaminoalkyl, alkenylaminoalkyl, alkoxyalkylaminoalkyl, heterocycloalkylalkylaminoalkyl,
d. aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)-aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl; or
e. heteroaryl, heteroarylalkyl, alkylheteroaryl, heteroarylalkylamino, heteroarylalkylaminoalkyl, arylalkyloxy or arylsulfonyl optionally substituted with up to three groups selected from CN, halogen and alkyl;
wherein any of the foregoing groups can be independently substituted with up to three groups selected from formyl, OH, halogen, C₁₋₆ alkoxy, amino, monoalkylamino, dialkylamino, hydroxyalkyl, arylalkyl, alkyl, aryl, heteroaryl, alkenyl, alkyiyl, heteroarylalkyl, CN, perhaloalkyl, monoalkylaminoalkyl, dialkylaminoalkyl, thiol, thioalkoxy, carboxyl, amido, amidino, NO₂, NO₃, perhaloalkoxy, S-alkyl, arylalkyloxy, S-arylalkyl, azido, hydrazino, hydroxylamino, sulfoxyl, sulfonyl, sulfide, disulfide, aryl optionally substituted with up to three halogen atoms, and urea optionally substituted with aryl wherein said aryl is optionally substituted with up to three halogen atoms;
n is 1 to 4;
p is 0 to 2;
R₄ is H, alkyl optionally substituted with C₁₋₆ alkoxy, allyl, alkoxyalkyl, heterocycloalkylalkyl, arylalkyl optionally substituted with up to three groups selected from dialkylamino, C₁₋₆ alkoxy, perhaloalkyl and halogen, heteroarylalkyl, monoalkylaminoalkyl, or dialkylaminoalkyl; wherein said alkyl is optionally substituted with C₁₋₆ alkoxy; and said arylalkyl is optionally substituted with up to three groups selected from dialkylamino, C₁₋₆ alkoxy, perhaloalkyl and halogen;
R₅ is H or alkyl;
or R₄ and R₅, together with the nitrogen atom to which they are attached, can form a heterocycloalkyl ring which can optionally be substituted with up to three alkyl groups;
R₇ and R₈ are independently H, NH₂, alkyl, alkoxy, aryl, heteroaryl, arylalkyl, heteroarylalkyl or heterocycloalkyl, wherein said aryl group can optionally be substituted with up to three groups selected from alkoxy, alkyl, perhaloalkyl, halogen and aryl;
R₂ is heteroaryl, arylalkyl, alkyl, formyl, —C(═O)NH₂, or —NHR₆;
R₆ is H, formyl, alkyl, alkenyl, alkynyl, arylalkyl, heterocycloalkyl, alkylsulfonyl, arylsulfonyl, —C(═O)NH₂, —C(═O)-alkyl, heteroarylalkyl, —C(═O)-alkylaminoalkyl, —C(═O)-aryl, arylalkanoylalkyl, heterocycloalkylalkyl, aryloxyalkyl, monoalkylaminoalkyl, dialkylaminoalkyl, allyl or urea;
wherein:
said alkyl, alkenyl or alkynyl groups can be optionally substituted with up to three groups selected from OH, halogen and C₁₋₆ alkoxy;
said arylalkyl is optionally substituted with up to three groups selected from OH, alkyl, perhaloalkyl, halogen, C₁₋₆ alkoxy, monoalkylamino, dialkylamino and hydroxyalkyl;
said heterocycloalkyl is optionally substituted with up to three groups selected from arylalkyl, alkyl, OH, halogen and C₁₋₆ alkoxy;
said arylsulfonyl is optionally substituted with up to three groups selected from CN, halogen, alkyl, OH, C₁₋₆ alkoxy, monoalkylamino, dialkylamino and hydroxyalkyl;
said —C(═O)-alkyl is optionally substituted with up to three groups selected from OH, halogen, perhaloalkyl and C₁₋₆ alkoxy;
said —C(═O)-aryl is optionally substituted with up to three groups selected from OH, alkyl, perhaloalkyl, halogen, C₁₋₆ alkoxy, monoalkylamino, dialkylamino and hydroxyalkyl
said heterocycloalkylalkyl is optionally substituted with up to three groups selected from OH, arylalkyl, alkyl, halogen and C₁₋₆ alkoxy;
said aryloxyalkyl is optionally substituted with up to three groups selected from OH, halogen, C₁₋₆ alkoxy, monoalkylamino, dialkylamino and hydroxyalkyl; and
said urea is optionally substituted with aryl, wherein said aryl is optionally substituted with up to three groups selected from OH, halogen, C₁₋₆ alkoxy, monoalkylamino, dialkylamino and hydroxyalkyl; and
R₉ is H or alkyl.

2. The method of claim 1 wherein R₁ is —C(═O)NR₄R₅.

3. The method of claim 1 wherein R₂ is NHR₆.

4. The method of claim 1 wherein R₁ is —C(═O)NR₄R₅ and R₂ is NHR₆.

5. The method of claim 4 wherein R₄ is H, alkyl, allyl, alkoxyalkyl, heterocycloalkylalkyl, arylalkyl, heteroarylalkyl, monoalkylaimnoalkyl or dialkylaminoalkyl, wherein said arylalkyl can be optionally substituted with up to three groups selected from halogen, haloalkyl, perhaloalkyl, C₁₋₆ alkoxy and dialkylamino.

6. The method of claim 4 wherein R₆ is alkyl, arylalkyl optionally substituted with up to three halogen atoms, heteroarylalkyl, N-alkanoylaminoalkyl, or heterocycloalkylalkyl.

7. The method of claim 4 wherein R₆ is alkyl, arylalkyl optionally substituted with up to three groups selected from halogen and C₁₋₆ alkoxy, heteroarylalkyl, N-alkanoylaminoalkyl, or heterocycloalkylalkyl.

8. The method of claim 1 wherein R₁ is —C(═O)NR₄R₅, where R₄ is alkyl, heteroarylalkyl, or heterocycloalkylalkyl.

9. The method of claim 8 wherein R₄ is alkyl

10. The method of claim 8 wherein R₄ is heteroarylalkyl.

11. The method of claim 8 wherein R₄ is heterocycloalkylalkyl

12. The method of claim 1 wherein R₂ is NHR₆, where R₆ is alkyl, arylalkyl optionally substituted with to up to three groups selected from halogen and C₁₋₆ alkoxy, heteroarylalkyl, or N-alkanoylaminoalkyl.

13. The method of claim 12 wherein R₆ is alkyl.

14. The method of claim 12 wherein R₆ is arylalkyl optionally substituted with up to three groups selected from halogen and C₁₋₆ alkoxy.

15. The method of claim 12 wherein R₆ is heteroarylalkyl.

16. The method of claim 12 wherein R₆ is N-alkanoulaminoalkyl.

17. The method of claim 1 wherein R₁ is —C(═O)NR₄R₅, where R₄ is alkyl, heteroarylalkyl, or heterocycloalkylalkyl; and R₂ is NHR₆, where R₆ is alkyl, arylalkyl optionally substituted with up to three halogen atoms, heteroarylalkyl, or N-alkanoylaminoalkyl.

18. The method of claim 17 wherein R₄ is heteroarylalkyl; and R₆ is alkyl or arylalkyl optionally substituted with up to three halogen atoms.

19. The method of claim 18 wherein R₆ is alkyl.

20. The method of claim 18 wherein R₆ is arylalkyl optionally substituted with up to three halogen atoms.

21. The method of claim 20 wherein said arylalkyl is phenylalkyl.

22. The method of claim 17 wherein R₄ heterocycloalkylalkyl; and R₆ is alkyl.

23. The method of claim 22 wherein said heterocycloalkylalkyl is pyrrolidino-alkyl.

24. The method of claim 17 wherein R₄ is alkyl; and R₆ is alkyl, arylalkyl optionally substituted with up to three halogen atoms, heteroarylalkyl, or N-alkanoylaminoalkyl.

25. The method of claim 24 wherein R₆ is alkyl.

26. The method of claim 24 wherein R₆ is arylalkyl optionally substituted with up to three halogen atoms.

27. The method of claim 26 wherein said arylalkyl is phenylalkyl.

28. The method of claim 24 wherein R₆ is heteroarylalkyl.

29. The method of claim 28 wherein said heteroarylalkyl is furanyl-alkyl.

30. The method of claim 24 wherein R₆ is N-alkanoylaminoalkyl.

31. The method of claim 1 wherein R₁ is halogen, alkyl, —C(═O)NH₂, or NO₂.

32. The method of claim 1 wherein R₂ is NHR₆ wherein R₆ is alkyl optionally substituted with dialkylamino, aryloxyalkyl, arylalkyl optionally substituted with up to three groups selected from C₁₋₆ alkoxy, halogen and OH, arylsulfonyl optionally substituted with up to three groups selected from CN and alkyl, —C(═O)aryl optionally substituted with up to three groups selected from CN and halogen, —C(═O)alkyl, heterocycloalkyl optionally substituted with up to three alkyl groups, or urea optionally substituted with aryl, said aryl being optionally substituted with up to three halogen atoms.

33. The method of claim 1 wherein R₁ is halogen, alkyl, —C(═O)NH₂, or NO₂; and R₂ is NHR₆ wherein R₆ is alkyl optionally substituted with dialkylamino, aryloxyalkyl, arylalkyl optionally substituted with up to three groups selected from C₁₋₆ alkoxy, halogen and OH, arylsulfonyl optionally substituted with up to three groups selected from CN and alkyl, —C(═O)aryl optionally substituted with up to three groups selected from CN and halogen, —C(═O)alkyl, heterocycloalkyl optionally substituted with up to three alkyl groups, or urea optionally substituted with aryl, said aryl being optionally substituted with up to three halogen atoms.

34. The method of claim 23, wherein R₁ is halogen, and R₆ is alkyl, aryloxyalkyl, or arylalkyl.

35. The method of claim 34 wherein R₆ is alkyl.

36. The method of claim 34 wherein R₆ is aryloxyalkyl.

37. The method of claim 36 wherein said aryloxyalkyl is phenoxyalkyl.

38. The method of claim 34 wherein R₆ is arylalkyl.

39. The method of claim 38 wherein said arylalkyl is phenylalkyl.

40. The method of claim 33, wherein R₁ is alkyl, and R₆ is arylsulfonyl optionally substituted with up to three groups selected from CN and alkyl, —C(═O)aryl optionally substituted with up to three groups selected from CN and halogen, urea optionally substituted with aryl, wherein said aryl is optionally substituted with up to three halogen atoms, —C(═O)alkyl, arylalkyl optionally substituted with up to three groups selected from halogen and OH, or alkyl optionally substituted with dialkylamino.

41. The method of claim 40 wherein R₆ is arylsulfonyl optionally substituted with up to three groups selected from CN and alkyl.

42. The method of claim 41 wherein said arylsulfonyl is phenylsulfonyl.

43. The method of claim 40 wherein R₆ is —C(═O)aryl optionally substituted with up to three groups selected from CN and halogen.

44. The method of claim 43 wherein said R₆ is —C(═O)phenyl optionally substituted with up to three groups selected from CN and halogen.

45. The method of claim 40 wherein R₆ is urea optionally substituted with aryl, wherein said aryl is optionally substituted with up to three halogen atoms.

46. The method of claim 45 wherein R₆ phenyl optionally substituted with up to three halogen atoms.

47. The method of claim 40 wherein R₆ is —C(═O)alkyl.

48. The method of claim 40 wherein R₆ is arylalkyl optionally substituted with up to three groups selected from halogen and OH.

49. The method of claim 40 wherein R₆ is phenylalkyl optionally substituted with up to three groups selected from halogen and OH.

50. The method of claim 40 wherein R₆ is alkyl optionally substituted with dialkylamino.

51. The method of claim 33, wherein R₁ is —C(═O)NH₂; and R₆ is arylalkyl.

52. The method of claim 51 wherein R₆ is phenylalkyl

53. The method of claim 33, wherein R₁ is NO₂, and R₆ is alkyl, arylalkyl optionally substituted with up to three C₁₋₆ alkoxy groups, or heterocycloalkyl optionally substituted with alkyl.

54. The method of claim 53 wherein R₆ is alkyl.

55. The method of claim 53 wherein R₆ is arylalkyl optionally substituted with up to three C₁₋₆ alkoxy groups.

56. The method of claim 55 wherein R₆ is phenylalkyl optionally substituted with up to three C₁₋₆ alkoxy groups.

57. The method of claim 53 wherein R₆ is heterocycloalkyl optionally substituted with alkyl.

58. The method of claim 57 wherein said heterocycloalkyl is piperidinyl.

59. The method of claim 1 wherein the compound is N-(4-methoxybenzyl)-6-methyl-2,3 4,9-tetrahydro-1H-carbazol-1-amine, 3-fluoro-N-(6-methyl-2,3,4,9-tetrahydro-1H-carbazol-1-yl)benzamide, N-bicyclo[2.2.1]hept-2-yl-6-nitro-2,3,4,9-tetrahydro-1H-carbazol-1-amine, 6-chloro-N-(4-fluorobenzyl)-2,3,4,9-tetrahydro-1H-carbazol-1-amine, 2-cyano-N-(6-methyl-2,3,4,9-tetrahydro-1H-carbazol-1-yl)benzamide, 6-bromo-N-cyclohexyl-2,3,4,9-tetrahydro-1H-carbazol-1-amine, 4-methyl-N-(6-methyl-2,3,4,9-tetrahydro-1H-carbazol- 1-yl)benzenesulfonamide, 6-bromo-N-(2-phenylethyl)-2,3,4,9-tetrahydro-1H-carbazol-1-amine, or N-(6-methyl-2,3,4,9-tetrahydro-1H-carbazol-1-yl)-3-(trifluoromethyl)benzamide.

60. A compound, stereoisomer, or pharmaceutically acceptable salt having the Formula II: 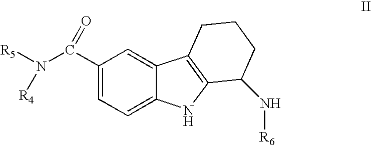
wherein:
R₄ and R₅ are each independently H, alkyl, allyl, alkoxyalkyl, heterocycloalkylalkyl, arylalkyl, heteroarylalkyl, monoalkylaminoalkyl, or dialkylaminoalkyl; wherein said alkyl is optionally substituted with C₁₋₆ alkoxy; and said arylalkyl is optionally substituted with up to thess groups selected from dialkylamino, C₁₋₆ alkoxy, perhaloalkyl and halogen;
or said R₄ and said R₅, together with the nitrogen atom to which they are attached, can form a heterocycloalkyl ring which can optionally be substituted with up to three alkyl groups; and
R₆ is alkyl, heteroarylalkyl, N-alkanoylaminoalkyl, heterocycloalkylalkyl, or arylalkyl optionally substituted with up to three groups selected from halogen and C₁₋₆ alkoxy.

61. The compound of claim 60 wherein R₄ is alkyl, heteroarylalkyl, or heterocycloalkylalkyl.

62. The compound of claim 60 wherein R₄ is alkyl.

63. The compound of claim 60 wherein R₄ is heteroarylalkyl.

64. The compound of claim 60 wherein R₄ is heterocycloalkylalkyl

65. The compound of claim 60 wherein R₆ is alkyl, arylalkyl optionally substituted with up to three groups selected from halogen and C₁₋₆ alkoxy, heteroarylalkyl, or N-alkanoylaminoalkyl.

66. The compound of claim 60 wherein R₆ is alkyl.

67. The compound of claim 60 wherein R₆ is arylalkyl optionally substituted with up up to three groups selected from halogen and C₁₋₆ alkoxy.

68. The compound of claim 60 wherein R₆ is heteroarylalkyl.

69. The compound of claim 60 wherein R₆ is N-alkanoylaminoalkyl.

70. The compound of claim 60 wherein R₄ is alkyl, heteroarylalkyl, or heterocycloalkylalkyl; and R₆ is alkyl, arylalkyl optionally substituted with up to three groups selected from halogen and C₁₋₆ alkoxy, heteroarylalkyl, or N-alkanoylaminoalkyl.

71. The compound of claim 60 wherein R₄ is heteroarylalkyl; and R₆ is alkyl or arylalkyl optionally substituted with up to three groups selected from halogen and C₁₋₆ alkoxy.

72. The compound of claim 71 wherein R₆ is alkyl.

73. The compound of claim 71 wherein R₆ is arylalkyl optionally substituted with up to three groups selected from halogen and C₁₋₆ alkoxy.

74. The compound of claim 73 wherein said arylalkyl is phenylalkyl.

75. The compound of claim 60 wherein R₄ heterocycloalkylalkyl; and R₆ is alkyl.

76. The method of claim 75 wherein said heterocycloalkylalkyl is pyrrolidino-alkyl.

77. The compound of claim 60 wherein R₄ is alkyl; and R₆ is alkyl, arylalkyl optionally substituted with up to three groups selected from halogen and C₁₋₆ alkoxy, heteroarylalkyl, or N-alkanoylaminoalkyl.

78. The compound of claim 77 wherein R₆ is alkyl.

79. The compound of claim 77 wherein R₆ is arylalkyl optionally substituted up to three groups selected from halogen and C₁₋₆ alkoxy.

80. The compound of claim 79 wherein said arylalkyl is phenylalkyl.

81. The compound of claim 77 wherein R₆ is heteroarylalkyl.

82. The compound of claim 81 wherein said heteroarylalkyl is furanyl-alkyl.

83. The compound of claim 77 wherein R₆ is N-alkanoylaminoalkyl.

84. The compound of any of claims 60-83 wherein R₅ is H.

85. The compound of claim 60 wherein R₅ is H, and R₄ and R₆ are selected in accordance with the following table:

| Com- | | |
|---|---|---|
| pound | R₄ | R₆ |
| 1 | phenylmethyl | cyclohexyl |
| 2 | cyclohexylmethyl | cyclohexyl |
| 3 | cyclohexyl | cyclohexyl |
| 4 | ethyl | cyclohexyl |
| 5 | allyl | cyclohexyl |
| 6 | isopropyl | cyclohexyl |
| 7 | methyl | cyclohexyl |
| 8 | 2-methoxyethyl | cyclohexyl |
| 9 | tetrahydrofuran-2- | cyclohexyl |
| | ylmethyl | |
| 10 | 3-phenylpropyl | cyclohexyl |
| 11 | 2-phenylethyl | cyclohexyl |
| 12 | 2-(4-fluorophenyl)ethyl | cyclohexyl |
| 13 | 4- | cyclohexyl |
| | trifluoromethylphenyl- | |
| | methyl | |
| 14 | 4-methoxyphenylmethyl | cyclohexyl |
| 15 | thien-2-yl-methyl | cyclohexyl |
| 16 | 2-oxopyrrolidin-1- | cyclohexyl |
| | ylpropyl | |
| 17 | pyridin-3-yl-methyl | cyclohexyl |
| 18 | (4-dimethylamino)phenyl- | cyclohexyl |
| | methyl | |
| 19 | pyridin-3-yl-methyl | 2-(4- |
| | | fluorophenyl)eth-1-yl |
| 20 | 2-(pyrrolidin-1- | cyclohexyl |
| | yl)ethyl | |
| 21 | ethyl | phenylmethyl |
| 22 | pyridin-3-yl-methyl | butyl-1-yl |
| 23 | pyridin-3-yl-methyl | hexyl-1-yl |
| 24 | pyridin-4-yl-methyl | cyclohexyl |
| 25 | pyridin-3-yl-methyl | 4- |
| | | methylcyclohex-1-yl |
| 26 | pyridin-3-yl-methyl | 2-(4- |
| | | chlorophenyl)eth-1-yl |
| 27 | pyridin-3-yl-methyl | cyclohexyl |
| 28 | ethyl | furan-2-yl-methyl |
| 29 | ethyl | 2-(4- |
| | | chlorophenyl)eth-1-yl |
| 30 | ethyl | 2-(4- |
| | | fluorophenyl)eth-1-yl |
| 31 | ethyl | —CH₂—CH₂—NH—C(═O)CH₃ |
| 32 | ethyl | hex-1-yl |
| 33 | ethyl | 3-phenyl-prop-1-yl |
| 34 | H | 2-phenyl-eth-1-yl |
| 35 | ethyl | 4-phenyl-but-1-yl |
| 36 | ethyl | cyclohexyl |
| 37 | pyridin-3-yl-methyl | cyclohexylmethyl |
| 38 | pyridin-3-yl-methyl | furan-2-yl-methyl |
| 39 | ethyl | phenylmethyl |

86. The compound of claim 60 wherein R₃ is H.

87. A compound, stereoisomer, or pharmaceutically acceptable salt of Formula I: 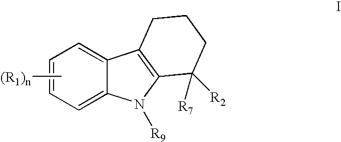
wherein:
each R₁ is independently
a. H, halogen, formyl, carbamoyl, carbamoylamino, carbamoyloxy, NO₂, amino, azido, hydrazino, hydroxylainino, sulfoxyl, sulfonyl, sulfide, disulfide, an ether having 2 to 10 carbon atoms and 1 to 6 oxygen or sulfur atoms;
b. alkyl, alkenyl, alkynyl, perhaloalkyl, alkoxy, alkoxyalkyl, —C(═O) alkyl, —OC(═O)alkyl, —C(═O)alkoxy, alkylsulfonyl, —C(═O) alkylamino, —C(═O) alkylaminoalkyl, —C(═O)NR₄R₅, —C(═O)NR₄R₆, —NHC(═O)R₇, —C(═O)R₈, monoalkylaminoalkyl, dialkylaminoalkyl, perhaloalkoxy, S-alkyl, urea optionally substituted with aryl wherein said aryl is optionally substituted with up to three halogen atoms;
c. heterocycloalkyl, heterocycloalkylamino, heterocycloalkylaminoalkyl, heterocycloalkylalkyl, monoalkylaminoalkyl, dialkylamninoalkyl, alkenylaminoalkyl, alkoxyalkylaminoalkyl, heterocycloalkylalkylaminoalkyl;
d. aryl, arylalkyl, alkylaryl, arylalkylamino, arylalkylaminoalkyl, arylsulfonyl, arylalkylsulfonyl, -arylalkanoylalkyl, —C(═O)aryl, —OC(═O)aryl, —C(═O)aryloxy, —C(═O)arylalkoxy, —C(═O)arylamino, aryloxyalkyl, arylalkanoylalkyl, —C(═O)arylalkyl, —OC(═O)arylalkyl, —C(═O)arylalkyloxy, arylalkanoylalkyl; or
e. heteroaryl, heteroarylalkyl, alkylheteroaryl, heteroarylalkylamino, heteroarylalkylaminoalkyl, arylalkyloxy, arylsulfonyl optionally substituted with up to three groups selected from CN, halogen and alkyl;
wherein any of the foregoing groups can be independently substituted with up to three groups selected from formyl, OH, halogen, C₁₋₆ alkoxy, amino, monoalkylamino, dialkylamino, hydroxyalkyl, arylalkyl, alkyl, aryl, heteroaryl, alkenyl, alkynyl, heteroarylalkyl, CN, perhaloalkyl, monoalkylaminoalkyl, dialkylaminoalkyl, thiol, thioalkoxy, carboxyl, amido, amidino, NO₂, NO₃, perhaloalkoxy, S-alkyl, arylalkyloxy, S-arylalkyl, azido, hydrazino, hydroxylamino, sulfoxyl, sulfonyl, sulfide, disulfide, aryl optionally substituted with up to three halogen atoms, and urea optionally substituted with aryl wherein said aryl is optionally substituted with up to three halogen atoms;
n is 1 to 4;
R₄ is H, alkyl optionally substituted with C₁₋₆ alkoxy, allyl, alkoxyalkyl, heterocycloalkylalkyl, heteroarylalkyl, monoalkylaminoalkyl, dialkylaminoalkyl or arylalkyl wherein said arylalkyl is optionally substituted with up to three groups selected from dialkylamino, C₁₋₆ alkoxy, perhaloalkyl and halogen;
R₅ is H or alkyl;
or R₄ and R₅, together with the nitrogen atom to which they are attached, can form a heterocycloalkyl ring which can optionally be substituted with up to three alkyl groups;
R₇ and R₈ are independently H, NH₂, alkyl, alkoxy, aryl, heteroaryl, arylalkyl, heteroarylalkyl or heterocycloalkyl, wherein said aryl group can optionally be substituted with up to three groups selected from alkoxy, alkyl, perhaloalkyl, halo and aryl;
R₂ is —NHR₆;
R₆ is cycloalkyl optionally substituted with up to three groups selected from OH, halogen, alkyl, amino, alkyl amino, cycloalkyl, heterocycloalkyl, aryl, heteroaryl, or C₁₋₆ alkoxy;
R₃ is H or alkyl; and
R₉ is H or alkyl.

88. The compound of claim 87 wherein R₆ is cycloalkyl optionally substituted with up to three groups selected from OH, halogen, alkyl, amino, alkyl amino, cycloalkyl, and aryl.

89. The method of claim 3 wherein R₆ is cycloalkyl optionally substituted with up to three groups selected from OH, halogen, alkyl, amino, alkyl amino, cycloalkyl, and aryl.

90. A pharmaceutical composition comprising a compound of any of claims 60-88.

91. A method for alleviating a symptom of a viral infection comprising administering to a patient suffering from said infection a compound of any of claims 60-88.

92. A method for alleviating a symptom of a viral infection comprising administering to a patient suffering from said infection a pharmaceutical composition comprising a compound of any of claims 60-88.

93. A method for alleviating a symptom of HCV comprising administering to a patient suffering from said infection a compound of any of claims 60-88.

94. A method for alleviating a symptom of HCV comprising administering to a patient suffering from said infection a pharmaceutical composition comprising a compound of any of claims 60-88.

95. A method for alleviating a symptom of SARS comprising administering to a patient suffering from said infection a compound of any of claims 60-88.

96. A method for alleviating a symptom of SARS comprising administering to a patient suffering from said infection a pharmaceutical composition comprising a compound of any of claims 60-88.

97. A method for treating HCV in a patient suffering therefrom, comprising administering to said patient a therapeutically effective amount of a substituted carbazole.

98. A method for treating HCV in a patient suffering therefrom, comprising administering to said patient a therapeutically effective amount of a substituted 1-amino-carbazole.

99. A method for treating HCV in a patient suffering therefrom, comprising administering to said patient a therapeutically effective amount of a substituted 1-amino-carbazole-6-carboxylic acid amide bearing at least one substituent on each of said 1-amino moiety and said carboxylic acid amide moiety.

100. A method for treating SARS in a patient suffering therefrom, comprising administering to said patient a therapeutically effective amount of a substituted carbazole.

101. A method for treating SARS in a patient suffering therefrom, comprising administering to said patient a therapeutically effective amount of a substituted 1-amino-carbazole.

102. A method for treating SARS in a patient suffering therefrom, comprising administering to said patient a therapeutically effective amount of a substituted 1-amino-carbazole-6-carboxylic acid amide bearing at least one substituent on each of said 1-amino moiety and said carboxylic acid amide moiety.

103. The method of claim 1 wherein said viral infection is HCV.

104. The method of claim 1 wherein said viral infection is SARS.
